(19) United States
(12) Patent Application Publication
(10) Pub. No.: US 2023/0108005 A1
(43) Pub. Date: Apr. 6, 2023

(54) FUNDUS IMAGE PROCESSING APPARATUS AND NON-TRANSITORY COMPUTER-READABLE STORAGE MEDIUM

(71) Applicant: NIDEK CO., LTD., Gamagori (JP)

(72) Inventors: Ryosuke SHIBA, Gamagori (JP); Tetsuya KANO, Gamagori (JP)

(73) Assignee: NIDEK CO., LTD., Gamagori (JP)

(21) Appl. No.: 17/955,772

(22) Filed: Sep. 29, 2022

(30) Foreign Application Priority Data
Sep. 30, 2021 (JP) 2021-160684

(51) Int. Cl. G06T 7/00; G06T 7/70; G06T 11/00; G06T 7/30; G06T 7/50; A61B 3/00; A61B 3/10; A61B 3/12

(52) U.S. Cl. CPC G06T 7/0012; G06T 7/70; G06T 11/005; G06T 7/30; G06T 7/50; A61B 3/0025; A61B 3/102; A61B 3/1233; G06T 2207/10101; G06T 2207/30041

(57) ABSTRACT

A fundus image processing apparatus including a controller configured to perform image acquisition processing of acquiring a three-dimensional tomographic image of the fundus of the subject eye, reference position setting processing of setting a reference position in a region of an optic nerve head in the two-dimensional measurement region in which the three-dimensional tomographic image was captured, radial pattern setting processing of setting a radial pattern with respect to the two-dimensional measurement region, image extraction processing of extracting a two-dimensional tomographic image in each of a plurality of lines of the radial pattern set in a redial pattern setting processing, and optic nerve head end detection processing of detecting a position of an end of the optic nerve head captured in the three-dimensional tomographic image.

### CROSS-REFERENCE TO RELATED APPLICATION

This application claims priority from Japanese Patent Application No. 2021-160684 filed on Sep. 30, 2021, the entire subject-matter of which is incorporated herein by reference.

### TECHNICAL FIELD

The present disclosure relates to a fundus image processing apparatus and a non-transitory computer-readable storage medium storing a fundus image processing program used for processing a fundus image of a subject eye.

### BACKGROUND ART

In recent years, a technique for identifying a specific site of a fundus by analyzing a fundus image of a subject eye has been proposed. For example, an ophthalmologic imaging apparatus disclosed in JP2018-083106A performs image processing (edge detection, Hough transform, or the like) on a front image of a fundus of a subject eye, to detect a position of an optic disk (hereinafter, also referred to as a “optic nerve head”) of the fundus captured in the front image.

It is possible to detect an approximate position of an optic nerve head from a front image of a fundus, but it is difficult to detect a position of an end of the optic nerve head with high accuracy. Here, it is also conceivable to detect the position of the end of the optic nerve head from a plurality of two-dimensional tomographic images configuring a three-dimensional tomographic image of the fundus. In this case, the plurality of two-dimensional tomographic images, configuring the three-dimensional tomographic image of the fundus, include an image in which the optic nerve head is captured and an image in which the optic nerve head is not captured. Therefore, the end of the optic nerve head may be erroneously detected from the two-dimensional tomographic image in which the optic nerve head is not captured. In a case where a plurality of two-dimensional tomographic images configuring the three-dimensional tomographic image of the fundus are processed, a processing amount also increases. As described above, it was difficult to appropriately detect the end of the optic nerve head captured in the fundus image with high accuracy.

### SUMMARY OF INVENTION

A typical object of the present disclosure is to provide a fundus image processing apparatus and a non-transitory computer-readable storage medium storing a fundus image processing program capable of appropriately detecting an end of an optic nerve head captured in a fundus image with high accuracy.

According to a first aspect of the present disclosure, there is provided a fundus image processing apparatus that processes a tomographic image of a fundus of a subject eye captured by an OCT apparatus, the fundus image processing apparatus including:

a controller configured to perform:
image acquisition processing of acquiring a three-dimensional tomographic image of the fundus of the subject eye, the three-dimensional tomographic image being captured by irradiating a two-dimensional measurement region extending in a direction intersecting an optical axis of OCT measurement light with the OCT measurement light;
reference position setting processing of setting a reference position in a region of an optic nerve head in the two-dimensional measurement region in which the three-dimensional tomographic image was captured;
radial pattern setting processing of setting a radial pattern with respect to the two-dimensional measurement region, the radial pattern being a line pattern extending radially around the reference position;
image extraction processing of extracting a two-dimensional tomographic image in each of a plurality of lines of the radial pattern set in the redial pattern setting processing, from the three-dimensional tomographic image; and
optic nerve head end detection processing of detecting a position of an end of the optic nerve head captured in the three-dimensional tomographic image, based on a plurality of the two-dimensional tomographic images extracted in the image extraction processing.

According to a second aspect of the present disclosure, there is provided a non-transitory computer-readable storage medium storing a fundus image processing program executed by a fundus image processing apparatus that processes a tomographic image of a fundus of a subject eye captured by an OCT apparatus, the fundus image processing program being executed by a controller of the fundus image processing apparatus to cause the fundus image processing apparatus to perform:
image acquisition processing of acquiring a three-dimensional tomographic image of the fundus of the subject eye, the three-dimensional tomographic image captured by irradiating a two-dimensional measurement region extending in a direction intersecting an optical axis of OCT measurement light with the OCT measurement light;
reference position setting processing of setting a reference position in a region of an optic nerve head in the two-dimensional measurement region in which the three-dimensional tomographic image was captured;
radial pattern setting processing of setting a radial pattern with respect to the two-dimensional measurement region, the radial pattern being a line pattern extending radially around the reference position;
image extraction processing of extracting a two-dimensional tomographic image in each of a plurality of lines of the radial pattern set in the radial pattern setting processing, from the three-dimensional tomographic image; and
optic nerve head end detection processing of detecting a position of an end of the optic nerve head captured in the three-dimensional tomographic image, based on a plurality of the two-dimensional tomographic images extracted in the image extraction processing.

According to the fundus image processing apparatus and the non-transitory computer-readable storage medium storing the fundus image processing program related to the above aspect of the present disclosure, an end of an optic nerve head captured in a fundus image is appropriately detected with high accuracy.

### BRIEF DESCRIPTION OF DRAWINGS

FIG. 1 is a block diagram showing a schematic configuration of a mathematical model construction apparatus 101, a fundus image processing apparatus 1, and OCT apparatuses 10A and 10B.

FIG. 2 is a block diagram showing a schematic configuration of an OCT apparatus 10.

FIG. 3 is an explanatory diagram for describing an example of a method of capturing a three-dimensional tomographic image.

FIG. 4 is a diagram showing an example of a two-dimensional tomographic image 42.

FIG. 5 is a diagram showing an example of a three-dimensional tomographic image 43 and a two-dimensional front image 45.

FIG. 6 is a diagram schematically showing a structure of a layer and a boundary in the fundus.

FIG. 7 is a flowchart showing a mathematical model construction processing performed by the mathematical model construction apparatus 101.

FIGS. 8A and 8B in combination show a flowchart showing fundus image processing performed by the fundus image processing apparatus 1.

FIG. 9 is an explanatory diagram for describing an example of an image alignment processing.

FIG. 10 is a diagram showing a state in which a reference position RP and a radial pattern 60 are set in a two-dimensional measurement region.

FIG. 11 is a diagram in which a two-dimensional tomographic image 64 extracted according to a radial pattern is compared with a probability map 65 of BMO output to the two-dimensional tomographic image 64.

FIG. 12 is an explanatory diagram for describing a method of detecting a position designated by a user as a position of the end of an optic nerve head.

FIG. 13 is a diagram showing an example of a two-dimensional front image on which a position 70 of a detected annular BMO is superimposed and displayed.

FIG. 14 is a diagram showing an example of a display method of two-dimensional tomographic images 75R and 75L and layer thickness graphs 76R and 76L.

FIGS. 15A and 15B in combination show a flowchart showing site identification processing performed by the fundus image processing apparatus 1.

FIG. 16 is a diagram schematically showing a relationship between the two-dimensional tomographic image 42 input to the mathematical model and one-dimensional regions A1 to AN in the two-dimensional tomographic image 42.

FIG. 17 is an explanatory diagram for describing an example of a method of identifying a second site based on a degree of deviation.

FIG. 18 is an explanatory diagram for describing an example of a method of detecting a position of a Cup 87 based on a position of a BMO 85.

### DESCRIPTION OF EMBODIMENTS

<Outline>

(First Aspect)

A controller of a fundus image processing apparatus exemplified in the present disclosure performs image acquisition processing, deviation degree acquisition processing, and site identification processing. In the image acquisition processing, the controller acquires a fundus image captured by a fundus image capturing apparatus. In the deviation degree acquisition processing, the controller inputs a fundus image into a mathematical model trained by a machine learning algorithm to acquire a probability distribution for identifying a first site of the fundus captured in the fundus image and acquire the degree of deviation of the acquired probability distribution with respect to a probability distribution in a case where the first site is accurately identified. In the site identification processing, the controller identifies a second site of the fundus which is different from the first site, based on the degree of deviation.

In the fundus, a state of the first site may change between a position where the second site is present and a position where the second site is not present. For example, a state of at least any one of a layer and a boundary of the fundus (first site) differs between a position where an optic nerve head (second site) is present and a position where the optic nerve head is not present (for example, around the optic nerve head). In general, a plurality of layers and boundaries are normally present around the optic nerve head, but specific layers and boundaries are missing at the position of the optic nerve head.

Here, a case is assumed in which a fundus image in which both the first site and the second site are captured is input to a mathematical model for identifying the first site. In this case, at a position where the first site is present, the first site is easily identified accurately, and thus the degree of deviation tends to decrease. On the other hand, in a case where the first site is missing at a position where the second site is present, the degree of deviation tends to increase. This tendency is likely to appear regardless of the presence or absence of an eye disease or the like.

Based on the above findings, the controller of the fundus image processing apparatus of the present disclosure identifies the second site based on the degree of deviation of a probability distribution in a case where the fundus image is input to the mathematical model for identifying the first site. As a result, the identification accuracy of the second site is improved regardless of the presence or absence of an eye disease or the like.

The degree of deviation will be described in more detail. In a case where the first site is identified with high accuracy by the mathematical model, an acquired probability distribution is likely to be biased. On the other hand, in a case where the identification accuracy of the first site by the mathematical model is low, an acquired probability distribution is less likely to be biased. Therefore, the degree of deviation between a probability distribution in a case where the first site is accurately identified and a probability distribution actually acquired changes according to a state of the first site. Therefore, according to the fundus image processing apparatus of the present disclosure, the second site can be identified with high accuracy regardless of the presence or absence of a disease, by using the degree of deviation in a case where a state of the first site changes between the position where the second site is present and the position where the second site is not present.

The degree of deviation may be output by the mathematical model. The controller may calculate the degree of deviation based on a probability distribution output by the mathematical model.

The degree of deviation may also be expressed as the uncertainty of identification of the first site performed by the mathematical model on the fundus image. The same result can also be obtained in a case where, for example, a reciprocal of a high certainty of identification (certainty) by the mathematical model is used as the degree of deviation.

The degree of deviation may include entropy (average amount of information) of the acquired probability distribution. The entropy represents the degree of uncertainty, messiness, and disorder. In the present disclosure, the entropy of a probability distribution output in a case where the first site is accurately identified is 0. The more difficult it is to identify the first site, the greater the entropy.

However, a value other than entropy may be employed as the degree of deviation. For example, at least any one of a standard deviation, a coefficient of variation, and a variance indicating the degree of scatter of the acquired probability distribution may be used as the degree of deviation. KL divergence or the like, which is a measure for measuring a difference between probability distributions, may be used as the degree of deviation. The maximum value of the acquired probability distribution may be used as the degree of deviation.

In the deviation degree acquisition processing, the degree of deviation may be acquired with at least any one of a plurality of layers and boundaries in the fundus captured in the fundus image as the first site. That is, the first site may be at least any one of a plurality of layers and boundaries between the layers in the fundus (hereinafter, also referred to as “layer/boundary”). As described above, a state of at least any one (first site) of the layers and boundaries of the fundus may differ between the position where the second site is present and the position where the second site is not present. Therefore, by acquiring the degree of deviation with the layer/boundary as the first site, it becomes easier to appropriately identify the second site based on the degree of deviation.

However, a site other than the layer/boundary in the fundus may be used as the first site. For example, a state of a fundus blood vessel may differ between the position where the second site is present and the position where the second site is not present. In this case, the degree of deviation may be acquired with the fundus blood vessel as the first site.

In a case where the first site is a layer/boundary, the controller may identify the optic nerve head (optic disk) in the fundus as the second site based on the degree of deviation, in the site identification processing. As described above, a state of at least any one of the layers and boundaries of the fundus differs between the position where the optic nerve head is present and the position where the optic nerve head is not present. Therefore, by setting the layer/boundary as the first site and the optic nerve head as the second site, the optic nerve head is appropriately detected based on the degree of deviation.

In a case where the layer/boundary is the first site and the optic nerve head is the second site, in the deviation degree acquisition processing, the degree of deviation may be acquired with at least any one of layers and boundaries at positions deeper than a nerve fiber layer (NFL) among the plurality of layers and boundaries of the fundus captured in the fundus image as the first site. At the position where the optic nerve head is present, the NFL is present, and layers and boundaries at positions deeper than the NFL are missing. That is, at the position where the optic nerve head is present, the degree of deviation related to identification of layers and boundaries at positions deeper than the NFL is larger than that at the position where the optic nerve head is not present. Therefore, by setting at least any one of the layers and the boundaries at the positions deeper than the NFL as the first site, the identification accuracy of the optic nerve head is further improved.

In the deviation degree acquisition processing, the degree of deviation may be acquired with at least any one of the NFL and the layers and the boundaries at the positions deeper than the NFL as the first site. In the site detection processing, a site in which the degree of deviation related to identification of a layer/boundary at a position deeper than the NFL is more than a first threshold value and the degree of deviation related to identification of the NFL is less than a second threshold value, may be detected as the optic nerve head. In this case, a position where a plurality of layers/boundaries including the NFL are missing due to the influence of a disease or the like, and the position where the optic nerve head is present are appropriately distinguished. Therefore, the identification accuracy of the optic nerve head is further improved.

The controller may acquire a three-dimensional tomographic image of the fundus as a fundus image, in the fundus image acquisition processing. The controller may further perform reference position setting processing, radial pattern setting processing, image extraction processing, and optic nerve head end detection processing. In the reference position setting processing, the controller sets a reference position in a region of the optic nerve head identified in the site identification processing in a two-dimensional measurement region in which a three-dimensional tomographic image is captured. In the radial pattern setting processing, the controller sets a radial pattern that is a line pattern extending radially around the reference position, with respect to the two-dimensional measurement region. In the image extraction processing, the controller extracts a two-dimensional tomographic image (a two-dimensional tomographic image that intersects each of the plurality of lines of the radial pattern) in each of the plurality of lines of the set radial pattern, from the three-dimensional tomographic image. In the optic nerve head end detection processing, the controller detects a position of the end of the optic nerve head captured in the three-dimensional tomographic image based on the plurality of extracted two-dimensional tomographic images.

In a case where the reference position is correctly set in the region of the optic nerve head in the reference position setting processing, the optic nerve head will always be included in all of the plurality of two-dimensional tomographic images extracted according to the radial pattern in the image extraction processing. Therefore, by detecting the position of the end of the optic nerve head based on the plurality of extracted two-dimensional tomographic images, a probability that the end of the optic nerve head is erroneously detected from the two-dimensional tomographic image in which the optic nerve head is not captured, is reduced. It is possible to suppress an excessive increase in an amount of image processing compared with a case of processing all of a plurality of two-dimensional tomographic images configuring a three-dimensional tomographic image. Therefore, the end of the optic nerve head is also detected with high accuracy, by using a result of identification of the optic nerve head site performed based on the degree of deviation.

In a case where the first site is a layer/boundary, the controller may identify the fovea in the fundus as the second site, based on the degree of deviation, in the site identification processing. A state of at least any one of the layers and boundaries of the fundus differs between a position where the fovea is present and a position where the fovea is not present. Therefore, by setting a layer/boundary as the first site and the fovea as the second site, the fovea can be appropriately detected based on the degree of deviation.

In a case where the layer/boundary is the first site and the fovea is the second site, in the deviation degree acquisition processing, the degree of deviation may be acquired with at least any one of layers and boundaries nearer to a surface side of the retina than the retinal pigment epithelium (RPE), among the plurality of layers and boundaries of the fundus captured in the fundus image, as the first site. At the position where the fovea is present, the RPE, the Bruch's membrane, and the like are present, and the layers and boundaries near to the surface side of the retina than the RPE are missing. That is, at the position where the fovea is present, the degree of deviation related to identification of the layer/boundary nearer to the surface side than the RPE is larger than that at the position where the fovea is not present. Therefore, by setting at least any one of the layers and the boundaries nearer to the surface side of the retina than the RPE as the first site, the identification accuracy of the fovea is further improved.

In the deviation degree acquisition processing, the degree of deviation may be acquired with both of at least one of the RPE and Bruch's membrane (hereinafter, simply referred to as “RPE/Bruch's membrane”), and at least any one of layers and boundaries nearer to the surface side than the RPE, as the first site. In the site detection processing, a site may be detected, as the fovea, in which the degree of deviation related to identification of the layer/boundary nearer to the surface side than the RPE is more than the first threshold value and the degree of deviation related to identification of the RPE/Bruch's membrane is less than the second threshold value. In this case, a position where a plurality of layers/boundaries including the RPE/Bruch's membrane are missing due to the influence of a disease or the like and a position where the fovea is present are appropriately distinguished. Therefore, the identification accuracy of the fovea is further improved.

The second site to be identified based on the degree of deviation is not limited to the optic nerve head and the fovea. The second site may be a site other than the optic nerve head and fovca in the fundus (for example, a macula or a fundus blood vessel). For example, at a position where the fundus blood vessel (second site) is present, measurement light is blocked by the fundus blood vessel, and an imaging state of a layer/boundary (first site) at a position deeper than the fundus blood vessel deteriorates. Therefore, at the position where the fundus blood vessel is present, the degree of deviation related to identification of the layer/boundary at the position deeper than the fundus blood vessel is larger than that at a position where the fundus blood vessel is not present. Therefore, the controller may identify a site in which the degree of deviation related to identification of at least any one of layers/boundaries at positions deeper than the fundus blood vessel is more than the threshold value, as a site in which the fundus blood vessel is present. The fundus image processing apparatus may identify a site of a disease existing in the fundus as the second site.

In the deviation degree acquisition processing, the controller may input a three-dimensional tomographic image of the fundus into the mathematical model to acquire a two-dimensional distribution of the degree of deviation in a case where the fundus is viewed from the front (that is, in a case where the fundus is viewed along an optical axis of imaging light of the fundus image). In the site identification processing, a position of the second site in a case where the fundus is viewed from the front may be identified based on the two-dimensional distribution of the degree of deviation. In this case, the second site is identified based on more data than in a case of identifying a two-dimensional position of the second site from the two-dimensional fundus image. Therefore, the identification accuracy of the second site is further improved.

A specific method of acquiring a two-dimensional distribution of the degree of deviation from a three-dimensional tomographic image may also be selected as appropriate. For example, the controller may input each of a plurality of two-dimensional tomographic images configuring the three-dimensional tomographic image into the mathematical model and arrange the degree of deviation acquired for each two-dimensional tomographic image in two dimensions, to acquire the two-dimensional distribution of the degree of deviation. The controller may input the entire three-dimensional tomographic image into the mathematical model to acquire a two-dimensional distribution of the degree of deviation. The tomographic images (three-dimensional tomographic image and two-dimensional tomographic image) may be captured by various devices such as an OCT apparatus or a Scheimpflug camera.

The controller may input a two-dimensional fundus image into the mathematical model to identify the second site in the fundus. For example, the controller may input a two-dimensional front image in a case where the fundus is viewed from the front into the mathematical model to identify a fundus blood vessel as the first site. The controller may detect the second site (for example, an optic nerve head) based on the acquired two-dimensional distribution of the degree of deviation. The two-dimensional front image may be an image captured by a fundus camera, an image captured by a scanning laser ophthalmoscope (SLO), or the like. The two-dimensional front image may be an Enface image generated based on data of a three-dimensional tomographic image captured by the OCT apparatus. The two-dimensional front image may be an image generated from motion contrast data obtained by processing a plurality of pieces of OCT data acquired from the same position at different times (so-called “motion contrast image”).

The controller may further perform front image acquisition processing and auxiliary identification result acquisition processing. In the front image acquisition processing, the controller acquires a two-dimensional front image in a case where the fundus of which the three-dimensional tomographic image is captured is viewed from the front. In the auxiliary identification result acquisition processing, the controller acquires an auxiliary identification result that is an identification result of the second site, which is performed based on the two-dimensional front image. The second site may be identified based on the degree of deviation and the auxiliary identification result. In this case, in addition to the degree of deviation obtained from the three-dimensional tomographic image, the auxiliary identification result based on the two-dimensional front image is also taken into consideration, and thus the second site is more appropriately identified.

A specific method of acquiring the auxiliary identification result may be selected as appropriate. For example, the auxiliary identification result may be a result of identifying the second site by performing image processing on the two-dimensional front image. In this case, the image processing may be performed by the controller of the fundus image processing apparatus, or may be performed by another device. The controller may acquire the auxiliary identification result by inputting the two-dimensional front image front image acquired in the front image acquisition processing into a mathematical model that outputs an identification result of the second site in the two-dimensional front image.

A specific method of identifying the second site based on the auxiliary identification result and the degree of deviation may also be selected as appropriate. For example, the controller may extract a part that is likely to include the second site, from the entire three-dimensional tomographic image acquired in the image acquisition processing, based on the auxiliary identification result. The controller may acquire the degree of deviation by inputting the extracted three-dimensional tomographic image into the mathematical model, and may identify the second site based on the acquired degree of deviation. In this case, an amount of processing by the mathematical model is reduced, and thus the second site can be identified more efficiently. The controller may identify the second site by adding the identification result based on the degree of deviation and the auxiliary identification result after performing any weighting. The controller may notify a user of a warning, an error, or the like in a case where a difference between the identification result based on the degree of deviation and the auxiliary identification result does not satisfy conditions.

The mathematical model may output a distribution of scores indicating a possibility of the second site, together with an identification result of the first site of the fundus captured in the fundus image. In the site identification processing, the second site may be identified based on the degree of deviation and the distribution of the scores. In this case, the second site is identified based on the distribution of the score of the second site and the degree of deviation, which is not easily affected by the presence or absence of an eye disease or the like. Therefore, the identification accuracy of the second site is further improved.

A specific method of identifying the second site based on both the degree of deviation and the distribution of scores may also be selected as appropriate. For example, the controller may identify the second site by adding an identification result based on the degree of deviation and an identification result based on the distribution of scores. In this case, the controller may add the identification results after performing any weighting. However, the controller may also identify the second site without using the distribution of scores of the second site.

(Second Aspect)

The controller of the fundus image processing apparatus exemplified in the present disclosure performs image acquisition processing, reference position setting processing, radial pattern setting processing, image extraction processing, and optic nerve head end detection processing. In the image acquisition processing, the controller acquires a three-dimensional tomographic image of a fundus of a subject eye captured by irradiating a two-dimensional measurement region extending in a direction intersecting an optical axis of OCT measurement light with the measurement light. In the reference position setting processing, the controller sets a reference position in a region of the optic nerve head in the two-dimensional measurement region in which the three-dimensional tomographic image is captured. In the radial pattern setting processing, the controller sets a radial pattern that is a line pattern extending radially around the reference position, with respect to the two-dimensional measurement region. In the image extraction processing, the controller extracts a two-dimensional tomographic image (a two-dimensional tomographic image that intersects each of the plurality of lines of the radial pattern) in each of the plurality of lines of the set radial pattern from the three-dimensional tomographic image. In the optic nerve head end detection processing, the controller detects a position of the end of the optic nerve head captured in the three-dimensional tomographic image based on the plurality of extracted two-dimensional tomographic images.

In a case where the reference position is correctly set in the region of the optic nerve head in the reference position setting processing, the optic nerve head will always be included in all of the plurality of two-dimensional tomographic images extracted according to the radial pattern in the image extraction processing. Therefore, by detecting the position of the end of the optic nerve head based on the plurality of extracted two-dimensional tomographic images, a probability that the end of the optic nerve head is erroneously detected from the two-dimensional tomographic image in which the optic nerve head is not captured is reduced. It is possible to suppress an excessive increase in an amount of image processing compared with a case of processing all of a plurality of two-dimensional tomographic images configuring a three-dimensional tomographic image. Therefore, the end of the optic nerve head is appropriately detected with high accuracy.

In a case where the tomographic image captured by the OCT apparatus is used for diagnosis, it is desirable that not only information regarding the optic nerve head but also various types of information such as a retina thickness can be obtained based on the tomographic image. Here, it is also conceivable to capture a plurality of two-dimensional tomographic images by actually scanning the fundus with measurement light along the radial pattern after setting the center of the radial pattern in the region of the optic nerve head. Even in this case, it seems that a position of the end of the optic nerve head can be detected from a plurality of captured two-dimensional tomographic images. However, it is difficult to obtain various types of information such as the retina thickness from a plurality of two-dimensional tomographic images captured according to the radial pattern. In contrast, the controller of the fundus image processing apparatus of the present disclosure may perform fundus analysis processing (for example, analysis of a thickness of a specific layer of the retina) on the three-dimensional tomographic image acquired in the image acquisition processing, in addition to the optic nerve head end detection processing. That is, according to the fundus image processing apparatus of the present disclosure, by using the three-dimensional tomographic image, it is possible not only to detect a position of the end of the optic nerve head with high accuracy but also to obtain an analysis result of the fundus.

The details of “the end of the optic nerve head” to be detected may be selected as appropriate. At least any one of, for example, a Bruch's Membrane Opening (BMO), the margin of the optic disk, and parapapillary atrophy (PPA) may be detected as the end of the optic nerve head.

Any of various apparatuses may function as the fundus image processing apparatus. For example, an OCT apparatus itself may function as the fundus image processing apparatus in the present disclosure. A device (for example, a personal computer or the like) capable of exchanging data with the OCT apparatus may function as the fundus image processing apparatus. Controllers of a plurality of devices may cooperate to perform processing.

The OCT apparatus may include a scanning unit. The scanning unit performs scanning, with measurement light applied to the tissue by an irradiation optical system, in a two-dimensional direction intersecting the optical axis. The three-dimensional tomographic image may be obtained by the scanning unit performing scanning, with a spot of the measurement light, in a measurement region, in the two-dimensional direction. In this case, a three-dimensional tomographic image is appropriately obtained by the OCT apparatus.

However, a configuration of the OCT apparatus may be changed. For example, the irradiation optical system of the OCT apparatus may simultaneously irradiate a two-dimensional region on the tissue of a subject with the measurement light. In this case, a light receiving element may be a two-dimensional light receiving element that detects an interference signal in the two-dimensional region on the tissue. That is, the OCT apparatus may acquire OCT data according to the principle of so-called full-field OCT (FF-OCT). The OCT apparatus may simultaneously irradiate an irradiation line extending in the one-dimensional direction on the tissue with the measurement light and perform scanning, with the measurement light, in a direction intersecting the irradiation line. In this case, the light receiving element may be a one-dimensional light receiving element (for example, a line sensor) or a two-dimensional light receiving element. That is, the OCT apparatus may acquire a tomographic image according to the principle of so-called line field OCT (LF-OCT).

The controller may further perform alignment processing of performing image alignment, in the direction along the optical axis of the OCT measurement light, of the three-dimensional tomographic image or the two-dimensional tomographic image extracted in the image extraction processing. The controller may detect a position of the end of the optic nerve head based on the two-dimensional tomographic image for which the image alignment has been performed. In this case, by performing image alignment, the deviation of the annular optic nerve head end in the direction along the optical axis of the OCT measurement light (tissue depth direction), is reduced. Therefore, the end of the optic nerve head is detected with higher accuracy.

The controller may further perform optic nerve head position detection processing of automatically detecting a position of the optic nerve head in a two-dimensional region intersecting the optical axis of the OCT measurement light, based on the image of the fundus. The controller may set a reference position at the automatically detected position of the optic nerve head. In this case, even in a case where the accuracy of automatic detection of the position of the optic nerve head is low, if the detected position is within the actual optic nerve head region, the end of the optic nerve head is appropriately detected in the subsequent optic nerve head end detection processing. Therefore, the detection processing is performed more smoothly.

In the optic nerve head position detection processing, a center position of the optic nerve head may be detected. In this case, there is a higher probability that a reference position will be within the region of the optic nerve head than in a case where a position other than the center of the optic nerve head is detected and set as a reference position.

A specific method of automatically detecting a position of the optic nerve head based on the image of the fundus may be selected as appropriate. As an example, at the position where the optic nerve head is present, the NFL is present, and layers and boundaries at positions deeper than the NFL are missing. Therefore, in a case where at least anyone of the layers and boundaries of the fundus (hereinafter simply referred to as a “layer/boundary”) captured in the three-dimensional tomographic image is detected by a mathematical model trained by using a machine learning algorithm, the uncertainty of detection of a layer/boundary at a position deeper than the NFL is high, at the position of the optic nerve head. Therefore, the controller may automatically detect the position (center position) of the optic nerve head based on the uncertainty in a case where the layer/boundary at the position deeper than the NFL is detected by the mathematical model. For example, the controller may detect a region where the uncertainty is equal to or more than a threshold value as a region of the optic nerve head, and detect the center of the detected region (for example, the center of gravity) as a center position of the optic nerve head.

The controller may automatically detect a position of the optic nerve head based on the two-dimensional front image in a case where the three-dimensional tomographic image is viewed from the front (a direction along the optical axis of the OCT measurement light). For example, the controller may perform known image processing on the two-dimensional front image, detect a region of the optic nerve head, and detect the center of the detected region as the center position of the optic nerve head. The controller may input the two-dimensional front image into the mathematical model that detects and outputs the position of the optic nerve head captured in the two-dimensional front image, to automatically detect a position (center position) of the optic nerve head. The two-dimensional front image may be a front image (so-called “Enface image” or the like) generated based on the three-dimensional tomographic image acquired in the image acquisition processing. The two-dimensional front image may be an image (for example, a fundus camera image or an SLO image) captured according to a principle different from the imaging principle of the three-dimensional tomographic image.

However, a method of setting a reference position may be changed. For example, the controller may set a reference position at a position designated by a user, in the two-dimensional measurement region. That is, the user may set the reference position by himself/herself. In this case, by setting the reference position in the region of the optic nerve head based on the user's experience or the like, the end of the optic nerve head is appropriately detected in the subsequent optic nerve head end detection processing. The controller may set a reference position at a position designated by the user, for example, in a case where the automatic detection of the position of the optic nerve head described above fails. The user may be made to set the reference position without performing the automatic detection of the position of the optic nerve head. For example, in a case where a position of the optic nerve head detected in the past is stored, a reference position may be set at the stored position of the optic nerve head. In this case, the processing of automatically detecting the position of the optic nerve head may be omitted.

In the optic nerve head end detection processing, a mathematical model trained by using a machine learning algorithm may be used. The mathematical model may be trained to output a detection result of the end of the optic nerve head captured in an input two-dimensional tomographic image. The controller may input the plurality of two-dimensional tomographic images extracted in the image extraction processing into the mathematical model and acquiring the position of the end of the optic nerve head output from the mathematical model, to detect a position of the end of the optic nerve head. In this case, the position of the end of the optic nerve head is automatically and appropriately detected from the plurality of two-dimensional tomographic images extracted according to the radial pattern.

The position of the end of the optic nerve head automatically detected by using the machine learning algorithm may be corrected according to an instruction from the user. For example, the controller may display the position of the end of the optic nerve head output from the mathematical model, on a display device, together with the two-dimensional tomographic image input to the mathematical model. The controller may correct the position of the end of the optic nerve head according to an instruction from the user who has checked the displayed position of the end of the optic nerve head. In this case, even in a case where the accuracy of automatic detection of the end of the optic nerve head is low, the position is appropriately corrected by the user. Therefore, the end of the optic nerve head is detected with higher accuracy.

However, a specific method of detecting a position of the end of the optic nerve head may be changed. For example, the controller may accept input of an instruction from the user in a state in which the two-dimensional tomographic image extracted in the image extraction processing is displayed on the display device. The controller may detect the position designated by the user as a position of the end of the optic nerve head. As described above, the two-dimensional tomographic image appropriately extracted according to the radial pattern always includes the optic nerve head. Therefore, the user can appropriately input (give an instruction for) the position of the end of the optic nerve head by checking the displayed two-dimensional tomographic image. As a result, the end of the optic nerve head is detected with high accuracy. The controller may automatically detect a position of the end of the optic nerve head by performing known image processing on the plurality of two-dimensional tomographic images extracted in the image extraction processing.

In the optic nerve head end detection processing, the controller may perform a smoothing processing on the detection results of the plurality of positions detected based on the plurality of two-dimensional tomographic images, to detect a position of the annular end of the optic nerve head. For example, due to the presence of a fundus blood vessel or the like, a position of the end of the optic nerve head in some two-dimensional tomographic images may be erroneously detected. In this case, the erroneously detected position of the annular end of the optic nerve head is separated from the appropriately detected position. In contrast, by performing a smoothing processing on the detection results of the plurality of detected positions, the influence of some of the erroneously detected positions is reduced. Therefore, the position of the annular end of the optic nerve head is more appropriately detected.

The controllcr may further perform optic nerve head center specifying processing of specifying a center position of the optic nerve head, based on the position of the optic nerve head end detected in the optic nerve head end detection processing. In this case, the center position of the optic nerve head is specified based on the position of the end of the optic nerve head detected with high accuracy. Therefore, the center position of the optic nerve head is specified with high accuracy.

A specific method of specifying a center position of the optic nerve head based on the detected position of the end of the optic nerve head may be selected as appropriate. For example, the controller may specify a detected position of the center of gravity of the annular optic nerve head end as a center position of the optic nerve head. The controller may fit an ellipse to the detected end of the optic nerve head and specify the center position of the fitted ellipse as a center position of the optic nerve head.

The controller may set the center position of the optic nerve head specified in the optic nerve head center specifying processing as a setting position of the reference position in the reference position setting processing, and perform the reference position setting processing, the radial pattern setting processing, the image extraction processing, and the optic nerve head end detection processing again. As the reference position becomes closer to a center position of the optic nerve head, a position of the end of the optic nerve head in each of the plurality of two-dimensional tomographic images extracted according to the radial pattern becomes more approximate, and thus the detection accuracy of the annular end of the optic nerve head becomes higher. Therefore, the accuracy of detection is further improved by detecting a position of the end of the optic nerve head again with the center position of the optic nerve head specified in the optic nerve head center specifying processing as a reference position.

The controller may further perform annular shape extraction processing and output processing. In the annular shape extraction processing, the controller extracts a two-dimensional tomographic image in an annular line pattern centered on the center position of the optic nerve head specified in the optic nerve head center specifying processing (that is, an image into which a tomographic image that intersects the annular line pattern in a cylindrical shape, is deformed in two dimensions), from the three-dimensional tomographic image. In the output processing, the controller outputs information regarding the two-dimensional tomographic image extracted in the annular shape extraction processing. In this case, a state of the tissue in the vicinity of the optic nerve head is appropriately observed with reference to the center position of the optic nerve head detected with high accuracy.

In a case where the information regarding the two-dimensional tomographic image extracted according to the annular line pattern is output, a specific method of outputting the information may be selected as appropriate. For example, the controller may display the extracted two-dimensional tomographic image on the display device. The controller may display a graph representing a thickness of a specific layer of the retina in the extracted two-dimensional tomographic image (for example, a thickness of the NFL or a thickness from the ILM to the NFL), on the display device. The controller may display at least anyone of a two-dimensional tomographic image of a patient and a graph, in comparison with disease-free normal eye data.

At the position where the fundus blood vessel is present, the end of the optic nerve head is difficult to be captured in the tomographic image, and thus the end of the optic nerve head is more likely to be erroneously detected. The controller may acquire information regarding the position of the fundus blood vessel in the measurement region in which the three-dimensional tomographic image is captured. The controller may adjust at least any one of an angle of the overall radial pattern, an angle of at least any one of the lines included in the radial pattern, a length of the line, the number of lines, and the like, to reduce an amount of overlap between the lines of the radial pattern and the fundus blood vessels as much as possible. In this case, the influence of the fundus blood vessels is reduced, and thus the detection accuracy of the end of the optic nerve head is further improved.

A method of acquiring information regarding a position of the fundus blood vessel may be selected as appropriate. For example, the controller may perform known image processing on a two-dimensional front image of the fundus (for example, an Enface image, an SLO image, or a fundus camera image), to detect a position of the fundus blood vessel. The controller may input the fundus image (a two-dimensional front image, a three-dimensional tomographic image, or the like) into the mathematical model trained by using the machine learning algorithm, to acquire a detection result of the fundus blood vessel output from the mathematical model. The controller may input an instruction, of the user who has checked the fundus image, on the position of the fundus blood vessel, to acquire information regarding the position of the fundus blood vessel.

The controller may adjust at least any one of the angle of the overall radial pattern, an angle of at least any one of the lines included in the radial pattern, a length of the line, the number of lines, and the like according to an instruction input by the user who has checked the fundus image. In this case, the user can appropriately set the radial pattern to reduce an amount of overlap between the lines of the radial pattern and the fundus blood vessels as much as possible.

The controller may also detect various structures of the fundus based on a detection result of the end of the optic nerve head. For example, in a case where the BMO is detected as the end of the optic nerve head, the controller may detect a position of an optic disk recess (Cup) based on the detected BMO. As an example, the controller may set a straight line parallel to a reference straight line passing through a detected pair of BMOs and separated, by a predetermined distance, from the reference straight line toward the surface side of the retina. The controller may detect a position where the set straight line and the internal limiting membrane (ILM) in the fundus image intersect, as a position of the Cup. The controller may detect the shortest distance between the detected BMO and the ILM in the fundus image as the minimum thickness (minimum rim width) of the nerve fiber layer.

### Embodiment

(Apparatus Configuration)

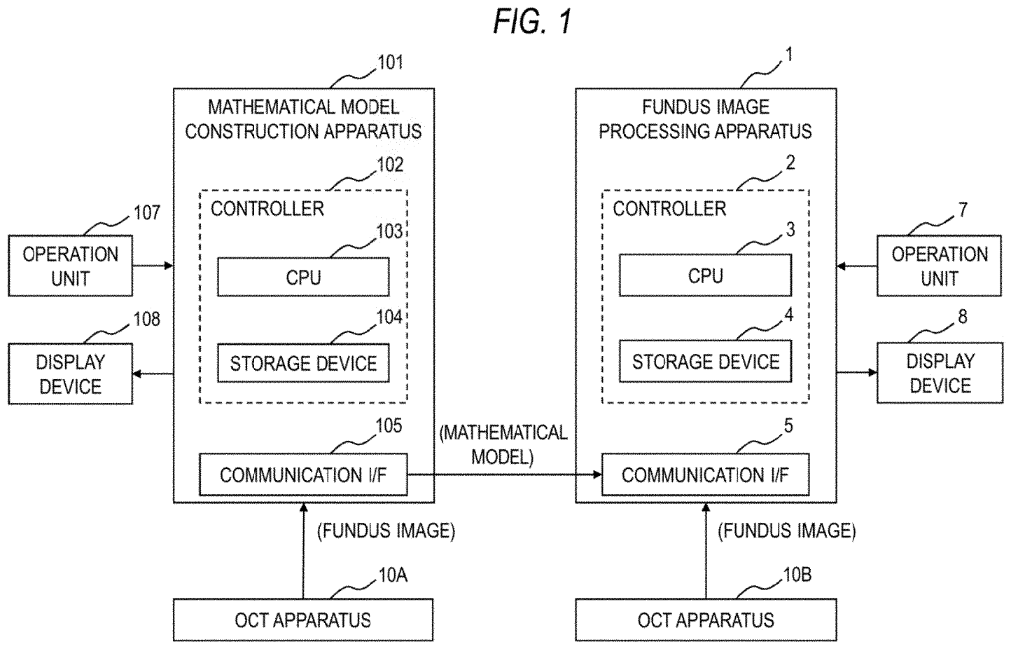

Hereinafter, one of the typical embodiments in the present disclosure will be described with reference to the drawings. As shown in FIG. 1, in the present embodiment, a mathematical model construction apparatus 101, a fundus image processing apparatus 1, and OCT apparatuses (fundus image capturing apparatuses) 10A and 10B are used. The mathematical model construction apparatus 101 constructs a mathematical model by training a mathematical model by using a machine learning algorithm. The constructed mathematical model identifies or detects a specific site captured in a fundus image based on the input fundus image. The fundus image processing apparatus 1 performs various processing by using results output from the mathematical model. The OCT apparatuses 10A and 10B function as fundus image capturing apparatuses capturing a fundus image (in the present embodiment, a tomographic image of the fundus) of a subject eye.

As an example, a personal computer (hereinafter, referred to as a “PC”) is used for the mathematical model construction apparatus 101 of the present embodiment. Although the details will be described later, the mathematical model construction apparatus 101 trains a mathematical model by using data of a fundus image of the subject eye (hereinafter, referred to as a “fundus image for training”) acquired from the OCT apparatus 10A and data indicating a first site (a site of the optic nerve head, in the present embodiment) of the subject eye of which the fundus image for training is captured. As a result, the mathematical model is constructed. However, a device that can function as the mathematical model construction apparatus 101 is not limited to a PC. For example, the OCT apparatus 10A may function as a mathematical model construction apparatus 101. Controllers of a plurality of devices (for example, a CPU of a PC and a CPU 13A of the OCT apparatus 10A) may cooperate to construct a mathematical model.

A PC is used for the fundus image processing apparatus 1 of the present embodiment. However, a device that can function as the fundus image processing apparatus 1 is not limited to a PC. For example, the OCT apparatus 10B or a server may function as the fundus image processing apparatus 1. Ina case where the OCT apparatus 10B functions as the fundus image processing apparatus 1, the OCT apparatus 10B can process a captured fundus image while capturing the fundus image. A portable terminal such as a tablet terminal or a smartphone may function as the fundus image processing apparatus 1. Controllers of a plurality of devices (for example, a CPU of a PC and a CPU 13B of the OCT apparatus 10B) may cooperate to perform various processing.

In the present embodiment, a case where a CPU is used as an example of a controller that performs various processing will be illustrated. However, it goes without saying that a controller other than the CPU may be used for at least some of various devices. For example, by employing a GPU as a controller, a processing speed may be increased.

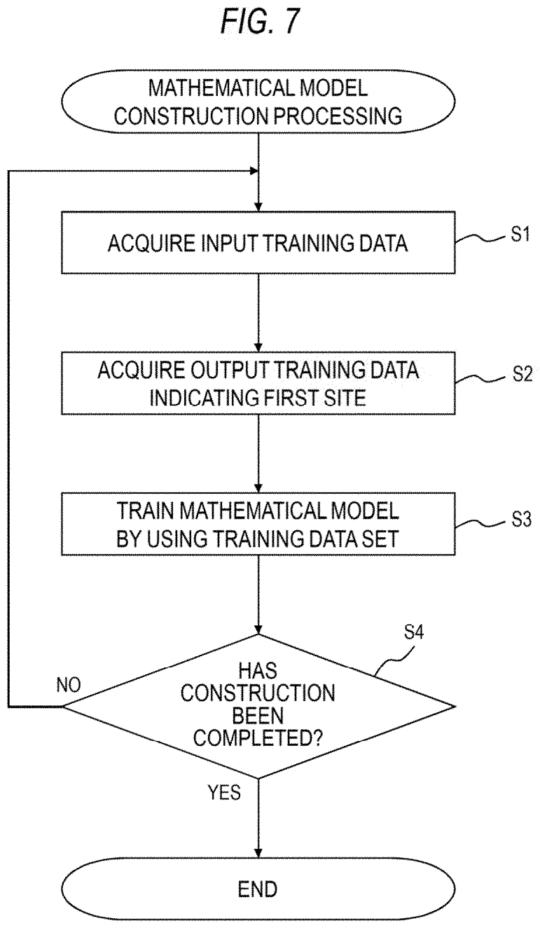

The mathematical model construction apparatus 101 will be described. The mathematical model construction apparatus 101 is provided in, for example, a manufacturer that provides the fundus image processing apparatus 1 or a fundus image processing program to a user. The mathematical model construction apparatus 101 includes a controller 102 that performs various control processing and a communication I/F 105. The controller 102 includes a CPU 103 that is a controller that performs control, and a storage device 104 that can store programs, data, and the like. The storage device 104 stores a mathematical model construction program for performing a mathematical model construction processing (refer to FIG. 7) described later. The communication I/F 105 connects the mathematical model construction apparatus 101 to other devices (for example, the OCT apparatus 10A and the fundus image processing apparatus 1).

The mathematical model construction apparatus 101 is connected to an operation unit 107 and a display device 108. The operation unit 107 is operated by a user in order for the user to input various instructions to the mathematical model construction apparatus 101. For the operation unit 107, at least any one of, for example, a keyboard, a mouse, and a touch panel may be used. A microphone or the like for inputting various instructions may be used together with the operation unit 107 or instead of the operation unit 107. The display device 108 displays various images. As the display device 108, various devices (for example, at least any one of, for example, a monitor, a display, and a projector) capable of displaying an image may be used. The “image” in the present disclosure includes both a still image and a moving image.

The mathematical model construction apparatus 101 may acquire data of a fundus image (hereinafter, may be simply referred to as a “fundus image”) from the OCT apparatus 10A. The mathematical model construction apparatus 101 may acquire data of the fundus image from the OCT apparatus 10A by using at least any one of, for example, wired communication, wireless communication, and a detachable storage medium (for example, a USB memory).

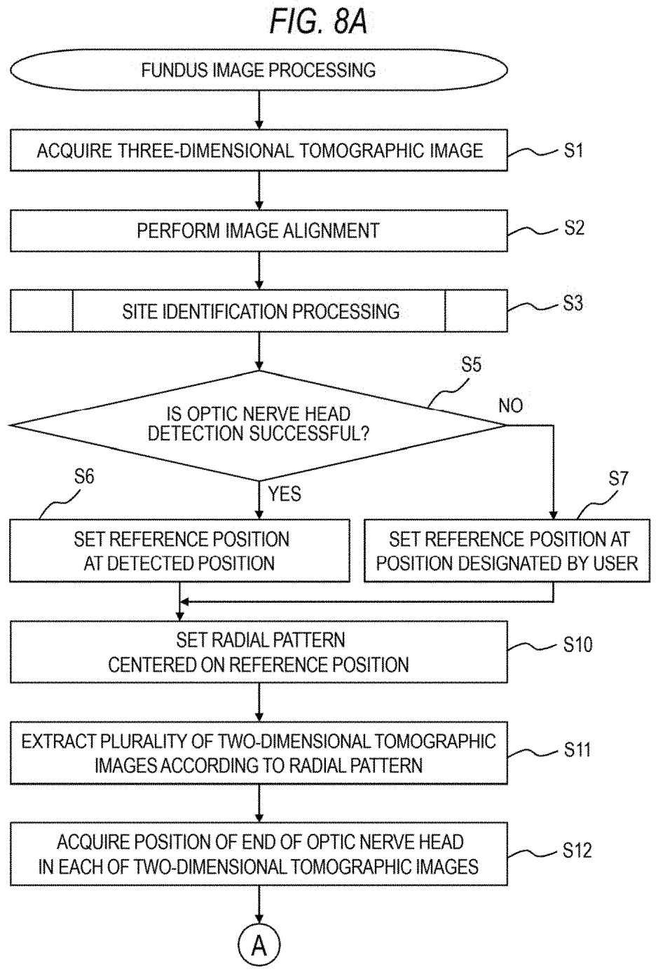

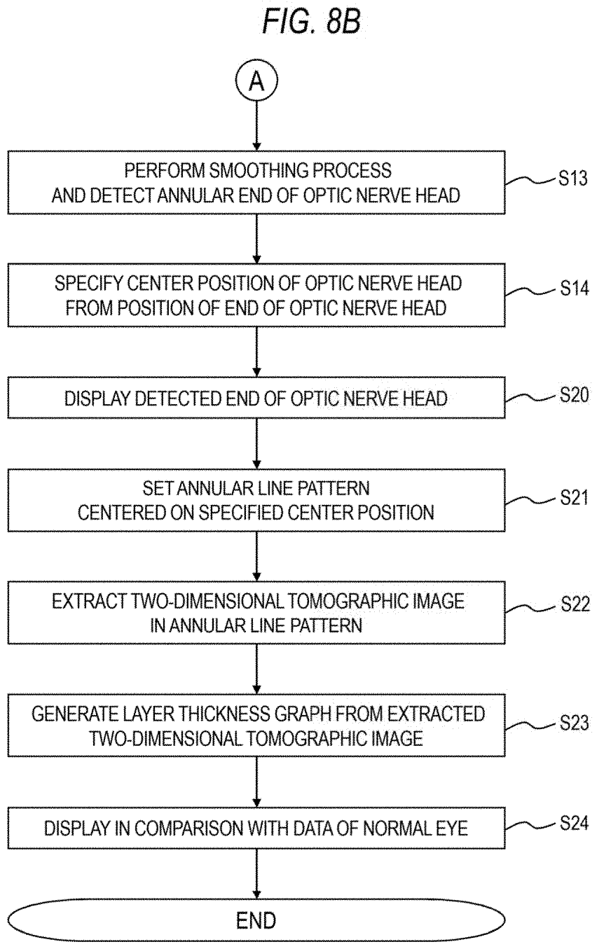

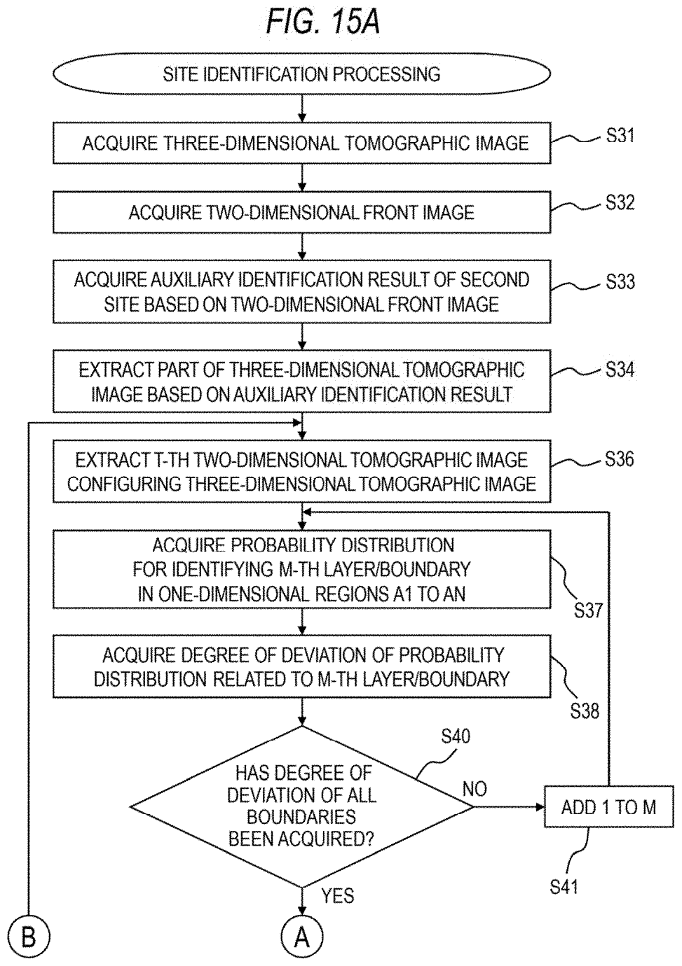

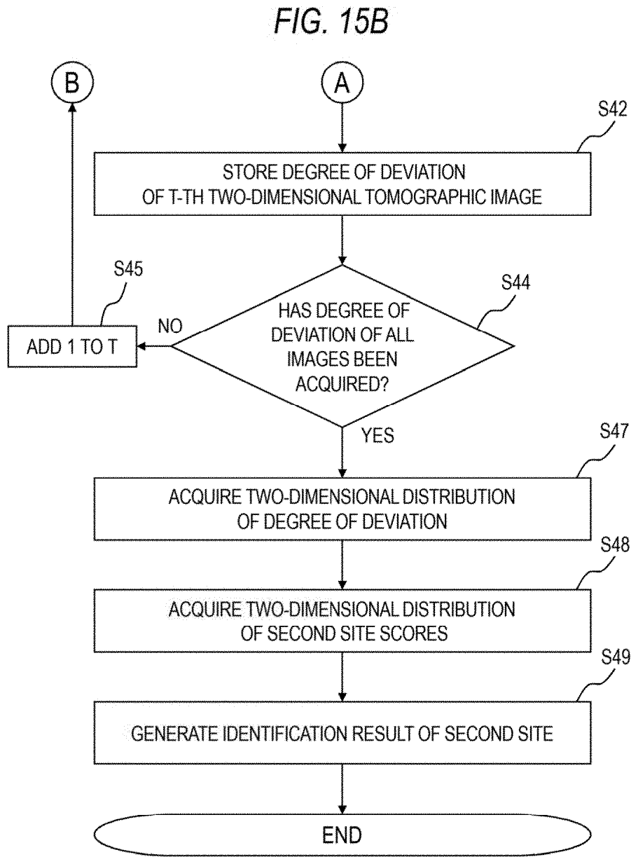

The fundus image processing apparatus 1 will be described. The fundus image processing apparatus 1 is provided in, for example, a facility (for example, a hospital or a health examination facility) for diagnosing or examining an examinee. The fundus image processing apparatus 1 includes a controller 2 that performs various control processing and a communication I/F 5. The controller 2 includes a CPU 3 which is a controller that performs control, and a storage device 4 that can store programs, data, and the like. The storage device 4 stores a fundus image processing program for performing fundus image processing (refer to FIGS. 8A and 8B) and a site identification processing (refer to FIGS. 15A and 15B), which will be described later. The fundus image processing program includes a program that realizes a mathematical model constructed by the mathematical model construction apparatus 101. The communication I/F 5 connects the fundus image processing apparatus 1 to other devices (for example, the OCT apparatus 10B and the mathematical model construction apparatus 101).

The fundus image processing apparatus 1 is connected to an operation unit 7 and a display device 8. As the operation unit 7 and the display device 8, various devices may be used in the same manner as the operation unit 107 and the display device 108 described above.

The fundus image processing apparatus 1 may acquire a fundus image (in the present embodiment, a three-dimensional tomographic image of the fundus) from the OCT apparatus 10B. The fundus image processing apparatus 1 may acquire a fundus image from the OCT apparatus 10B by using at least any one of, for example, wired communication, wireless communication, and a detachable storage medium (for example, a USB memory). The fundus image processing apparatus 1 may acquire a program or the like for realizing the mathematical model constructed by the mathematical model construction apparatus 101, via communication or the like.

The OCT apparatus 10 (10A, 10B) will be described. As an example, in the present embodiment, a case where the OCT apparatus 10A providing a fundus image to the mathematical model construction apparatus 101, and the OCT apparatus 10B providing a fundus image to the fundus image processing apparatus 1 are used, will be described. However, the number of OCT apparatuses used is not limited to two. For example, the mathematical model construction apparatus 101 and the fundus image processing apparatus 1 may acquire fundus images from a plurality of OCT apparatuses. The mathematical model construction apparatus 101 and the fundus image processing apparatus 1 may acquire fundus images from one common OCT apparatus.

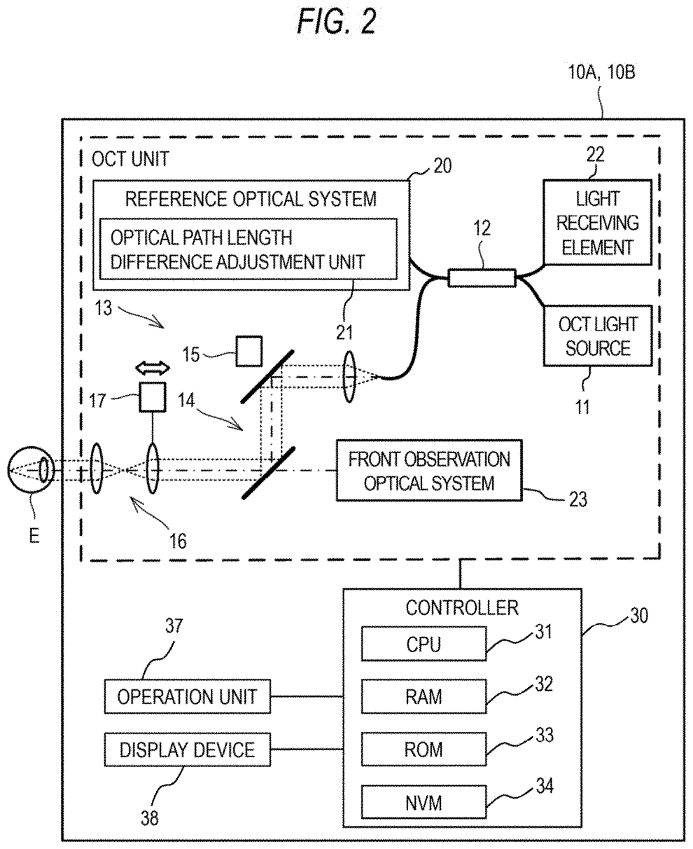

As shown in FIG. 2, the OCT apparatus 10 includes an OCT unit and a controller 30. The OCT unit includes an OCT light source 11, a coupler (light splitter) 12, a measurement optical system 13, a reference optical system 20, a light receiving element 22, and a front observation optical system 23.

The OCT light source 11 emits light (OCT light) for acquiring OCT data. The coupler 12 divides the OCT light emitted from the OCT light source 11 into measurement light and reference light. The coupler 12 of the present embodiment combines the measurement light reflected by a subject (in the present embodiment, the fundus of a subject eye E) and the reference light generated by the reference optical system 20, to interfere with each other. That is, the coupler 12 of the present embodiment serves as both a branch optical element that branches the OCT light into the measurement light and the reference light, and a multiplexing optical element that combines reflected light of the measurement light and the reference light.

The measurement optical system 13 guides the measurement light divided by the coupler 12 to the subject, and returns the measurement light reflected by the subject to the coupler 12. The measurement optical system 13 includes a scanning unit 14, an irradiation optical system 16, and a focus adjustment unit 17. By being driven by a drive unit 15, the scanning unit 14 can perform scanning with (deflect) the measurement light in a two-dimensional direction intersecting an optical axis of the measurement light. The irradiation optical system 16 is provided further toward the downstream side (that is, the subject side) of the optical path than the scanning unit 14, and irradiates the tissue of the subject with the measurement light. The focus adjustment unit 17 moves an optical member (for example, a lens) included in the irradiation optical system 16 in a direction along the optical axis of the measurement light, to adjust a focus of the measurement light.

The reference optical system 20 generates reference light and returns the reference light to the coupler 12. The reference optical system 20 of the present embodiment reflects the reference light divided by the coupler 12 by using a reflection optical system (for example, a reference mirror), to generate the reference light. However, a configuration of the reference optical system 20 may also be changed. For example, the reference optical system 20 may transmit the light incident from the coupler 12 without reflecting the incident light, to return the incident light to the coupler 12. The reference optical system 20 includes an optical path length difference adjustment unit 21 that changes an optical path length difference between the measurement light and the reference light. In the present embodiment, an optical path length difference is changed by moving the reference mirror in the optical axis direction. A configuration for changing an optical path length difference may be provided in the optical path of the measurement optical system 13.

The light receiving element 22 receives interference light between the measurement light and the reference light generated by the coupler 12, to detect an interference signal. In the present embodiment, the principle of Fourier domain OCT is employed. In the Fourier domain OCT, the spectral intensity (spectral interference signal) of the interference light is detected by the light receiving element 22, and a complex OCT signal is acquired by performing Fourier transform on the spectral intensity data. As an example of the Fourier domain OCT, any of spectral-domain-OCT (SD-OCT), swept-source-OCT (SS-OCT), and the like, may be employed. For example, time-domain-OCT (TD-OCT) may be employed.

In the present embodiment, the scanning unit 14 scans, with a spot of the measurement light, in a two-dimensional measurement region, and thus three-dimensional OCT data (three-dimensional tomographic image) is acquired. However, the principle of acquiring three-dimensional OCT data may also be changed. For example, three-dimensional OCT data may be acquired based on the principle of line field OCT (hereinafter, referred to as “LF-OCT”). In the LF-OCT, the measurement light is simultaneously applied on an irradiation line extending in the one-dimensional direction in the tissue, and the interference light between the reflected light of the measurement light and the reference light is received by a one-dimensional light receiving element (for example, a line sensor) or a two-dimensional light receiving element. In the two-dimensional measurement region, scanning with the measurement light is performed in a direction intersecting the irradiation line, and thus the three-dimensional OCT data is acquired. The three-dimensional OCT data may be acquired based on the principle of full-field OCT (hereinafter, referred to as “FF-OCT”). In the FF-OCT, the measurement light is applied to the two-dimensional measurement region on the tissue, and the interference light between the reflected light of the measurement light and the reference light is received by a two-dimensional light receiving element. In this case, the OCT apparatus 10 may not include the scanning unit 14.

The front observation optical system 23 is provided for capturing a two-dimensional front image of the tissue of the subject (in the present embodiment, the fundus of the subject eye E) in real time. The front observation image in the present embodiment is a two-dimensional front image in a case where the tissue is viewed from the direction (front direction) along the optical axis of the measurement light of the OCT. In the present embodiment, a scanning laser ophthalmoscope (SLO) is employed as the front observation optical system 23. However, for the configuration of the front observation optical system 23, a configuration other than an SLO (for example, an infrared camera that collectively irradiates a two-dimensional imaging range with infrared light to capture a front image), may be employed.

The controller 30 performs various types of control of the OCT apparatus 10. The controller 30 includes a CPU 31, a RAM 32, a ROM 33, and a nonvolatile memory (NVM) 34. The CPU 31 is a controller that performs various types of control. The RAM 32 temporarily stores various types of information. The ROM 33 stores a program executed by the CPU 31, various initial values, and the like. The NVM 34 is a non-transitory storage medium capable of storing storage contents even in a case where the power supply is cut off. The controller 30 is connected to an operation unit 37 and a display device 38. As the operation unit 37 and the display device 38, various devices may be used in the same manner as the operation unit 107 and the display device 108 described above.

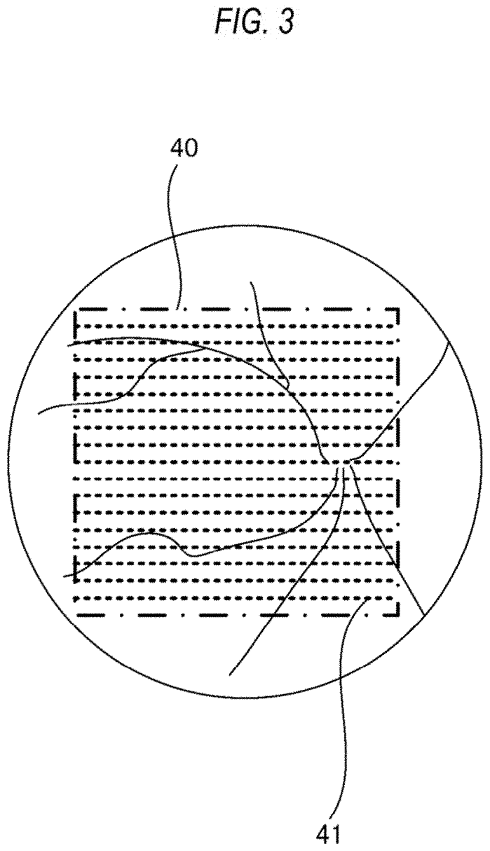

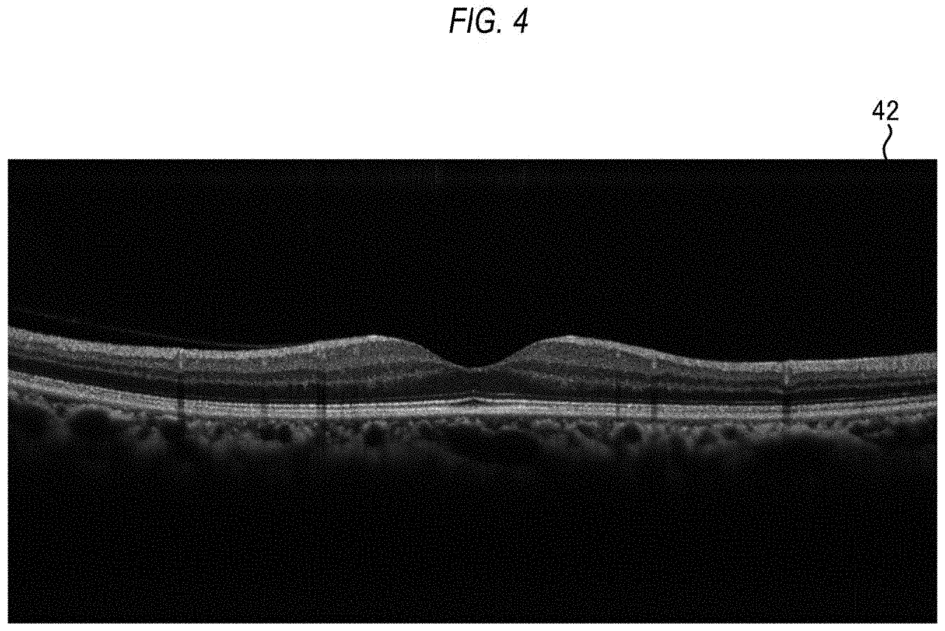

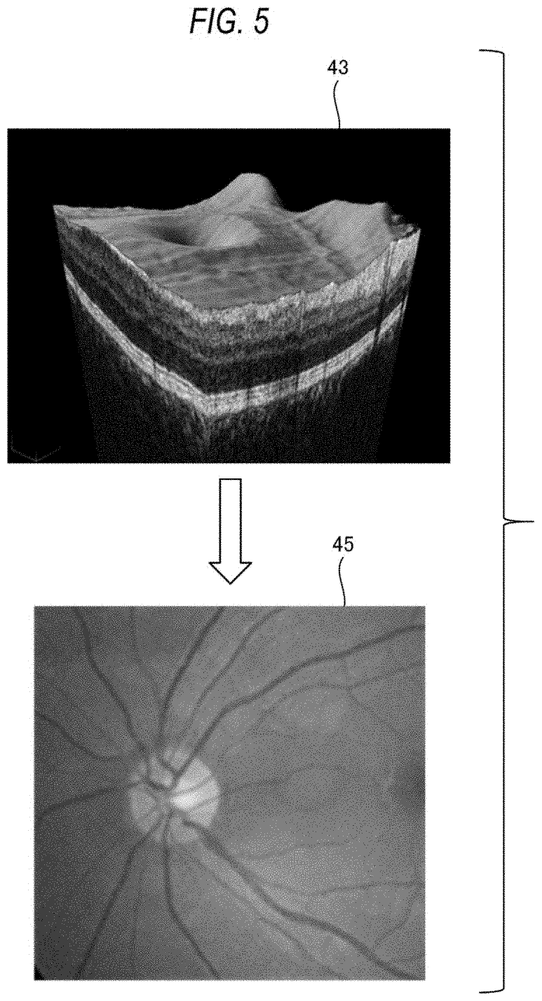

A method of capturing a fundus image in the present embodiment will be described. As shown in FIG. 3, the OCT apparatus 10 of the present embodiment sets a plurality of linear scanning lines (scan lines) 41 for performing scanning with spots in a two-dimensional measurement region 40 extending in a direction intersecting the optical axis of the OCT measurement light at equal intervals. The OCT apparatus 10 can capture a two-dimensional tomographic image 42 (refer to FIG. 4) of a cross section intersecting each scanning line 41 by performing scanning with the spot of measurement light on each scanning line 41. The two-dimensional tomographic image 42 may be an addition averaging image generated by performing an addition averaging processing on a plurality of two-dimensional tomographic images of the same site. The OCT apparatus 10 may acquire (capture) a three-dimensional tomographic image 43 (refer to FIG. 5) by arranging the plurality of two-dimensional tomographic images 42 captured for the plurality of scanning lines 41 in a direction orthogonal to the image region.

The OCT apparatus 10 may acquire (generate) an Enface image 45 that is a two-dimensional front image in a case where the tissue is viewed from the direction (front direction) along the optical axis of the measurement light, based on the captured three-dimensional tomographic image 43. In a case where the enface image 45 is acquired in real time, the front observation optical system 23 may be omitted. Data of the enface image 45 may be, for example, integrated image data in which luminance values are integrated in a depth direction (Z direction) at respective positions in the XY direction, integrated values of spectral data at respective positions in the XY direction, and luminance data at each position in the XY direction in a certain depth direction, or luminance data at each position in the XY direction in any layer of the retina (for example, the surface layer of the retina). The Enface image 45 may be obtained from a motion contrast image (for example, an OCT angiography image) obtained by acquiring a plurality of OCT signals from the same position in the tissue of the patient's eye at different times.

FIG. 1 will be referred to again. The OCT apparatus 10A connected to the mathematical model construction apparatus 101 can capture at least the two-dimensional tomographic image 42 (refer to FIG. 4) of the fundus of the subject eye. The OCT apparatus 10B connected to the fundus image processing apparatus 1 can capture the three-dimensional tomographic image 43 (refer to FIG. 5) of the fundus of the subject eye, in addition to the two-dimensional tomographic image 42 described above.

(Structure of Layer/Boundary of Fundus)

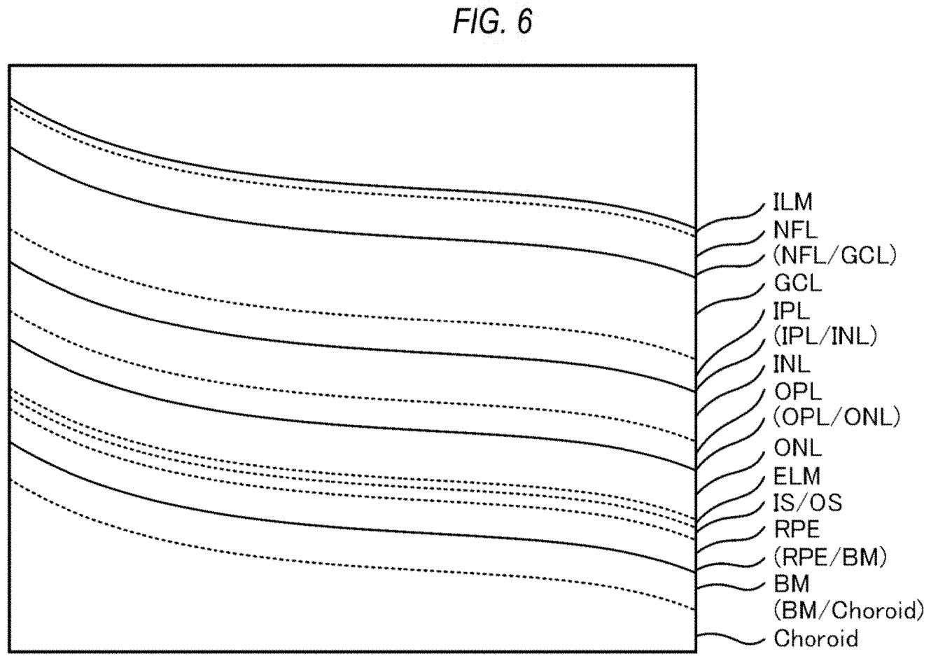

With reference to FIG. 6, a structure of layers in the fundus of the subject eye and a boundary between the layers adjacent to each other, will be described. FIG. 6 schematically shows a structure of the layer/boundary in the fundus. The upper side in FIG. 6 is a surface side of the retina of the fundus. That is, the depth of the layer/boundary increases toward the lower side in FIG. 6. In FIG. 6, parentheses are attached to the names of the boundaries between adjacent layers.

The layers of the fundus will be described. In the fundus, from the surface side (upper side in FIG. 6), an internal limiting membrane (ILM), a nerve fiber layer (NFL), a ganglion cell layer (GCL), an inner plexiform layer: (IPL), an inner nuclear layer (INL), an outer plexiform layer (OPL), an outer nuclear layer (ONL), an external limiting membrane (ELM), a junction between the photoreceptor inner and outer segment (IS/OS), a retinal pigment epithelium (RPE), a Bruch's membrane (BM), and a choroid, are present.

As boundaries that arc likely to appear in tomographic images, for example, NFL/GCL (a boundary between the NFL and the GCL), IPL/INL (a boundary between the IPL and the INL), and OPL/ONL (a boundary between the OPL and the ONL), RPE/BM (a boundary between the RPE and the BM), and BM/choroid (a boundary between the BM and the choroid), are present.

(Mathematical Model Construction Processing)

A mathematical model construction processing performed by the mathematical model construction apparatus 101 will be described with reference to FIG. 7. The mathematical model construction processing is performed by the CPU 103 according to the mathematical model construction program stored in the storage device 104.

In the following description, as an example, a case will be exemplified in which a mathematical model that outputs an identification result of at least any one (a specific layer/boundary that is the first site in the present embodiment) of a plurality of layers/boundaries captured in a fundus image, by analyzing an input two-dimensional tomographic image, is constructed. However, in the mathematical model construction processing, a mathematical model that outputs a result different from the identification result of the layer/boundary may be constructed. For example, a mathematical model that outputs a detection result of the end of the optic nerve head captured in the input two-dimensional tomographic image (details thereof will be described later) is also constructed by the mathematical model construction processing.

The mathematical model exemplified in the present embodiment is trained to output a distribution of scores indicating a probability that each site (each A scan image) in a two-dimensional tomographic image is the second site (the optic nerve head in the present embodiment), together with an identification result of the first site (specific layer/boundary in the present embodiment) captured in the input fundus image.

In the mathematical model construction processing, the mathematical model is constructed by training the mathematical model with a training data set. The training data set includes input side data (input training data) and output side data (output training data).

As shown in FIG. 7, the CPU 103 acquires data of a fundus image (two-dimensional tomographic image, in the present embodiment) captured by the OCT apparatus 10A as input training data (S1). Next, the CPU 103 acquires data indicating the first site of a subject eye of which the fundus image acquired in S1 is captured, as output training data (S2). The output training data in the present embodiment includes label data indicating a position of a specific layer/boundary captured in the fundus image. The label data may be generated, for example, by an operator operating the operation unit 107 while looking at the layers/boundaries in the fundus image. In the present embodiment, in order for the mathematical model to output a score indicating a probability of the second site (the optic nerve head in the present embodiment), label data indicating the second site in the fundus image is also included in the output training data.

Next, the CPU 103 performs training of the mathematical model using a training data set according to a machine learning algorithm (S3). As the machine learning algorithm, for example, a neural network, a random forest, boosting, and a support vector machine (SVM), are generally known.

The neural network is a technique that mimics the behavior of biological nerve cell networks. The neural network includes, for example, a feedforward neural network, a radial basis function (RBF) network, a spiking neural network, a convolutional neural network, a recursive neural network (a recurrent neural network, a feedback neural network, or the like), and a probabilistic neural network (a Boltzmann machine, a Basian network, or the like).

The random forest is a method of performing learning based on randomly sampled training data to generate a large number of decision trees. In a case where the random forest is used, the branch of plurality of decision trees learned in advance as a discriminator are traced, and an average (or a majority) of results obtained from the respective decision trees is taken.

The boosting is a method of generating a strong discriminator by combining a plurality of weak discriminators. The strong discriminator is constructed by sequentially learning a simple and weak discriminator.

The SVM is a method of constructing two classes of pattern discriminators by using a linear input element. The SVM learns parameters of the linear input element on the basis of, for example, a criterion (hyperplane separation theorem) of obtaining the margin maximizing hyperplane in which a distance to each data point is maximized from the training data.

The mathematical model refers to, for example, a data structure for predicting a relationship between input data (in the present embodiment, data of a two-dimensional tomographic image similar to the input training data) and output data (in the present embodiment, data of an identification result of the first site). The mathematical model is constructed by being trained with a training data set. As described above, the training data set is a set including input training data and output training data. For example, each piece of correlation data (for example, weights) between input and output is updated through training.

In the present embodiment, a multi-layered neural network is used as a machine learning algorithm. The neural network includes an input layer for inputting data, an output layer for generating data of an analysis result desired to be predicted, and one or more hidden layers between the input layer and the output layer. A plurality of nodes (also called units) are disposed in each layer. Specifically, in the present embodiment, a convolutional neural network (CNN) that is a kind of multi-layered neural network is used.

Other machine learning algorithms may be used. For example, generative adversarial networks (GAN) that utilize two competing neural networks may be employed as a machine learning algorithm.

The processing in S1 to S3 are repeatedly performed until the construction of the mathematical model is completed (S4: NO). In a case where the construction of the mathematical model is completed (S4: YES), the mathematical model construction processing is ended. In the present embodiment, a program and data for realizing the constructed mathematical model are incorporated in the fundus image processing apparatus 1.

(Fundus Image Processing)

Fundus image processing performed by the fundus image processing apparatus 1 will be described with reference to FIGS. 8A to 17. In the fundus image processing of the present embodiment, a plurality of two-dimensional tomographic images are extracted from a three-dimensional tomographic image according to a radial pattern, and the end of the optic nerve head is detected based on the plurality of extracted two-dimensional tomographic images. As an example, in the present embodiment, a case where a position of the Bruch's membrane opening (BMO) is detected as a position of the end of the optic nerve head will be exemplified. The fundus image processing of the present embodiment also includes a site identification processing (refer to S3 in FIG. 8A, and FIGS. 15A and 15B). In the site identification processing, the second site (the optic nerve head, in the present embodiment) different from the first site is identified, based on the degree of deviation of a probability distribution in a case where the mathematical model identifies the first site (a specific layer/boundary in the present embodiment). The CPU 3 of the fundus image processing apparatus 1 performs the fundus image processing shown in FIGS. 8A and 8B and the site identification processing shown in FIGS. 15A and 15B according to the fundus image processing program stored in the storage device 4.

As shown in FIG. 8A, the CPU 3 acquires a three-dimensional tomographic image of the fundus of the subject eye (S1). As described above, the three-dimensional tomographic image 43 (refer to FIG. 5) is captured by irradiating the two-dimensional measurement region 40 (refer to FIG. 3) with the OCT measurement light. The three-dimensional tomographic image 43 of the present embodiment is configured by arranging the plurality of two-dimensional tomographic images 42 (refer to FIG. 4).

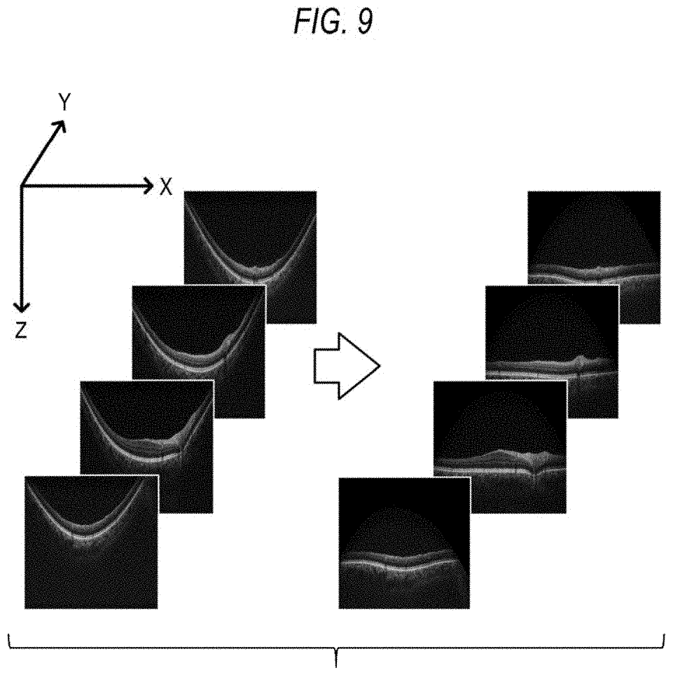

The CPU 3 performs image alignment, in the direction along the optical axis of the OCT measurement light (the Z direction in the present embodiment), of the three-dimensional tomographic image acquired in S1 (S2). In FIG. 9, some of the two-dimensional tomographic images included in the three-dimensional tomographic image before and after the image alignment is performed, are compared. The left side in FIG. 9 shows two-dimensional tomographic images before the image alignment is performed, and the right side in FIG. 9 shows two-dimensional tomographic images after the image alignment is performed. As shown in FIG. 9, by performing the image alignment, a deviation in the image including the optic nerve head in the Z direction is reduced. As a result, in the processing in S12 and S13 that will be described later, the end of the optic nerve head is detected with higher accuracy.

As an example, in S2 of the present embodiment, image alignment in the Z direction is performed between a plurality of two-dimensional tomographic images configuring the three-dimensional tomographic image. For each of the plurality of two-dimensional tomographic images configuring the three-dimensional tomographic image, image alignment is performed between a plurality of pixel arrays (in the present embodiment, a plurality of A-scan images extending in the Z direction) configuring the two-dimensional tomographic image.

Instead of the processing in S2, the image alignment in the Z direction may be performed for a plurality of two-dimensional tomographic images extracted in S11 that will be described later. In this case, the detection accuracy of the end of the optic nerve head is improved. The image alignment processing may be omitted.

Next, the CPU 3 performs the site identification processing (S3). The site identification processing is a processing of identifying a specific site in the two-dimensional measurement region 40 (refer to FIG. 3) of which a three-dimensional tomographic image is captured, based on an image of the fundus of the subject eye that is an examination target. The site identification processing (S3) of the present embodiment is performed as an automatic optic nerve head detection processing. The site identification processing (S3) of the present embodiment is a processing in the preparatory stage for detecting a position of the end of the optic nerve head with high accuracy in the processing that will be described later. Details of the site identification processing will be described later with reference to FIGS. 15A to 17.

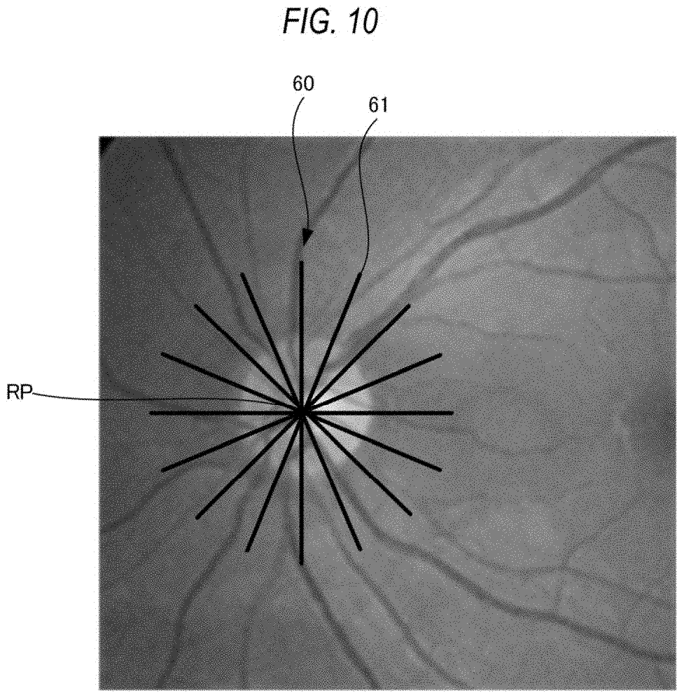

In a case where the automatic optic nerve head detection is successful (S5: YES), a reference position is set at a position of the detected optic nerve head (in the present embodiment, the center position of the optic nerve head automatically detected in S3) (S6). As shown in FIG. 10, a reference position RP serves as a reference for setting a radial pattern 60 that will be described later.

On the other hand, in a case where the automatic optic nerve head detection fails (S5: NO), the CPU 3 sets a reference position at a position designated by the user (S7). In the present embodiment, the CPU 3 receives input of an instruction from the user in a state in which the fundus image (for example, a two-dimensional front image) of the subject eye that is an examination target, is displayed on the display device 8. In a case where the user inputs an instruction for designating a position via the operation unit 7, the CPU 3 sets a reference position at the designated position. In some cases, a reference position may be set through the processing in S7 without performing the processing in S3, S5, and S6.

Next, the CPU 3 sets a radial pattern centered on the reference position in the two-dimensional measurement region 40 (S10). As shown in FIG. 10, in the processing in S10, a pattern of lines 61 extending radially around the reference position RP is set as the radial pattern 60. In a case where the reference position RP is correctly set in the region of the optic nerve head, all of the plurality of lines 61 configuring the radial pattern 60 pass through the end of the optic nerve head. As an example, in the radial pattern 60 shown in FIG. 10, sixteen lines 61 having the same length, with the reference position RP as one end, extend in the direction away from the reference position RP at the same intervals.

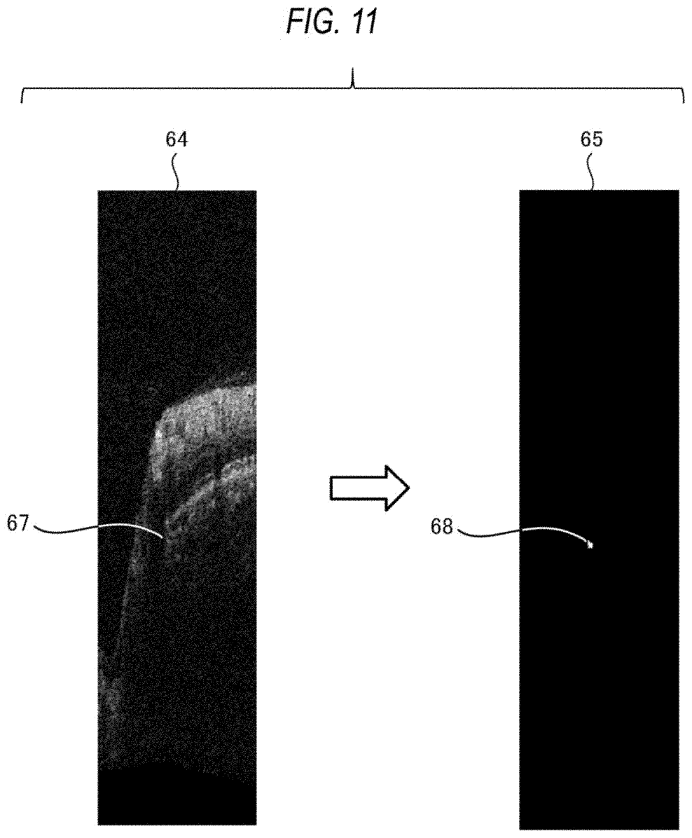

Next, the CPU 3 extracts a two-dimensional tomographic image 64 (refer to FIG. 1I) in each of the lines 61 of the radial pattern 60 set in S10, from the three-dimensional tomographic image acquired in S1 (S11). That is, the CPU 3 extracts a plurality of two-dimensional tomographic images 64 that intersect corresponding lines 61 of the radial pattern 60, from the three-dimensional tomographic image. In a case where the reference position RP is correctly set in the region of the optic nerve head, all of the two-dimensional tomographic images 64 extracted in S11 will include the end of the optic nerve head. A BMO 67 of the optic nerve head is captured in the two-dimensional tomographic image 64 shown in FIG. 11.

The CPU 3 acquires a position of the end of the optic nerve head (the BMO in the present embodiment) in each of the plurality of two-dimensional tomographic images 64 extracted in S11 (S12). In the present embodiment, the CPU 3 inputs the two-dimensional tomographic image 64 into the mathematical model. The mathematical model is trained by using a machine learning algorithm to output a detection result of a position of the BMO captured in the input two-dimensional tomographic image. Specifically, as shown in FIG. 11, in the mathematical model of the present embodiment, in a case where the two-dimensional tomographic image 64 is input, the mathematical model outputs a probability map 65 indicating a distribution of a probability that is a position of the BMO 67 in the region of the input two-dimensional tomographic image 64. In the probability map 65 shown in FIG. 11, a position 68 where the BMO 67 actually is present, is white indicating that a probability of the BMO 67 is high. The CPU 3 acquires a detection result (a position where the probability map 65 becomes maximum) of the position of the BMO output by the mathematical model, to detect the position of the BMO.

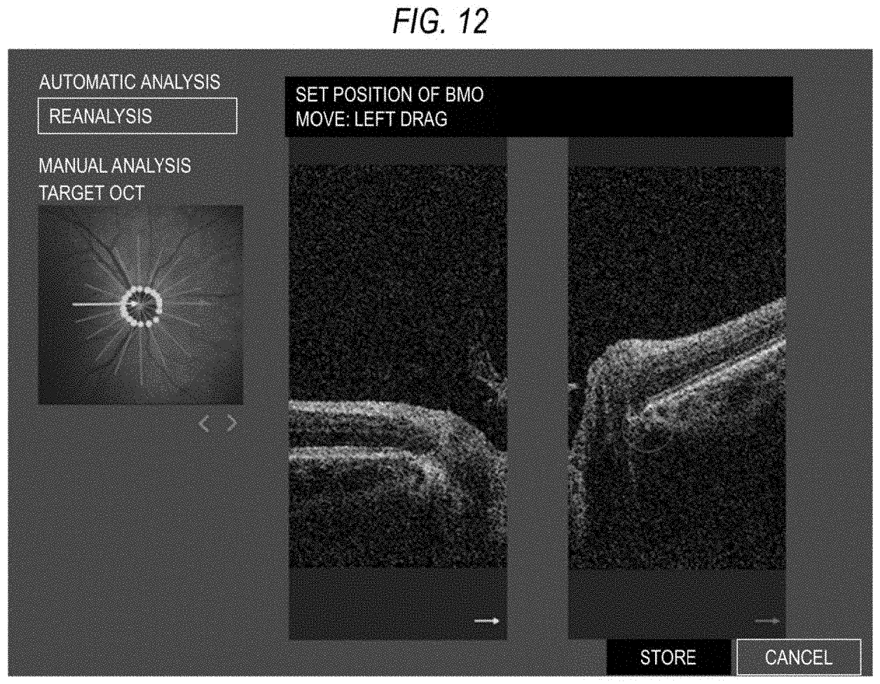

In the present embodiment, the position of the BMO automatically detected by using the machine learning algorithm is corrected according to an instruction from the user. Specifically, as shown in FIG. 12, the CPU 3 displays the position where the automatic detection result having the highest probability of the BMO is obtained on the two-dimensional tomographic image extracted in S11. In a case where the position displayed based on the automatic detection result is inaccurate, the user inputs an accurate BMO position via the operation unit 7 or the like. The CPU 3 detects the position input by the user as a position of the BMO. The CPU 3 may also detect a position designated by the user as a position of the BMO without using the machine learning algorithm.

Next, the CPU 3 performs a smoothing processing on the detection results of the plurality of positions detected based on the plurality of two-dimensional tomographic images 64, to detect a position of the annular end of the optic nerve head (an annular BMO in the present embodiment) (S13). As a result, even in a case where a position of the end is erroneously detected for some of the two-dimensional tomographic images 64, the influence of the erroneous detection is suppressed. As an example, in the present embodiment, a smoothing processing using a one-dimensional Gaussian filter is performed, on each dimension of XYZ of the detection results of the plurality of positions detected based on the plurality of two-dimensional tomographic images 64. A smoothing processing using a three-dimensional Gaussian filter may be performed on the plurality of probability maps 65, before a position of the BMO is detected. Elliptical fitting or the like for a plurality of detection results may be used for smoothing.

The CPU 3 specifies a center position of the optic nerve head based on the position of the end of the optic nerve head detected in S12 and S13 (S14). As an example, in the present embodiment, the CPU 3 specifies the detected position of the center of gravity of the annular BMO in the XY plane as the center position of the optic nerve head in the XY plane.

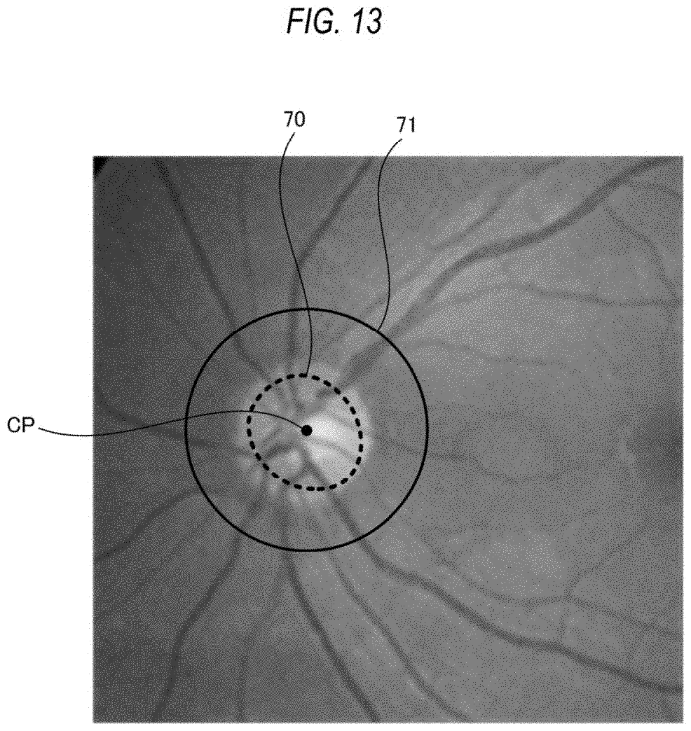

The CPU 3 displays the detected position of the end of the optic nerve head on the display device 8 (S20). In the present embodiment, as shown in FIG. 13, the detected position 70 of the annular BMO is superimposed and displayed on the two-dimensional front image of the fundus of the subject eye that is an examination target. Specifically, the CPU 3 performs spline interpolation on the detected positions of the plurality of BMOs in the XY plane, and displays contour lines of the BMOs. Therefore, the user can appropriately ascertain a two-dimensional position of the BMO.

Next, as shown in FIG. 13, the CPU 3 sets an annular line pattern 71 (a perfect annular shape in the present embodiment) centered on the center position CP of the optic nerve head specified in S14, with respect to the two-dimensional measurement region (S21). A diameter of the line pattern 71 is predetermined, but the diameter may be changed according to an instruction from the user.

The CPU 3 extracts, from the three-dimensional tomographic image acquired in S1, a two-dimensional tomographic image in the annular line pattern 71 set in S21 (that is, an image into which a tomographic image that intersects the annular line pattern 71 in a cylindrical shape deformed in two dimensions) (S22).

The CPU 3 processes the two-dimensional tomographic image extracted in S22 to generate a layer thickness graph representing a thickness of a specific layer of the retina (for example, a thickness of the NFL or a thickness from the ILM to the NFL) captured in the two-dimensional tomographic image (S23).

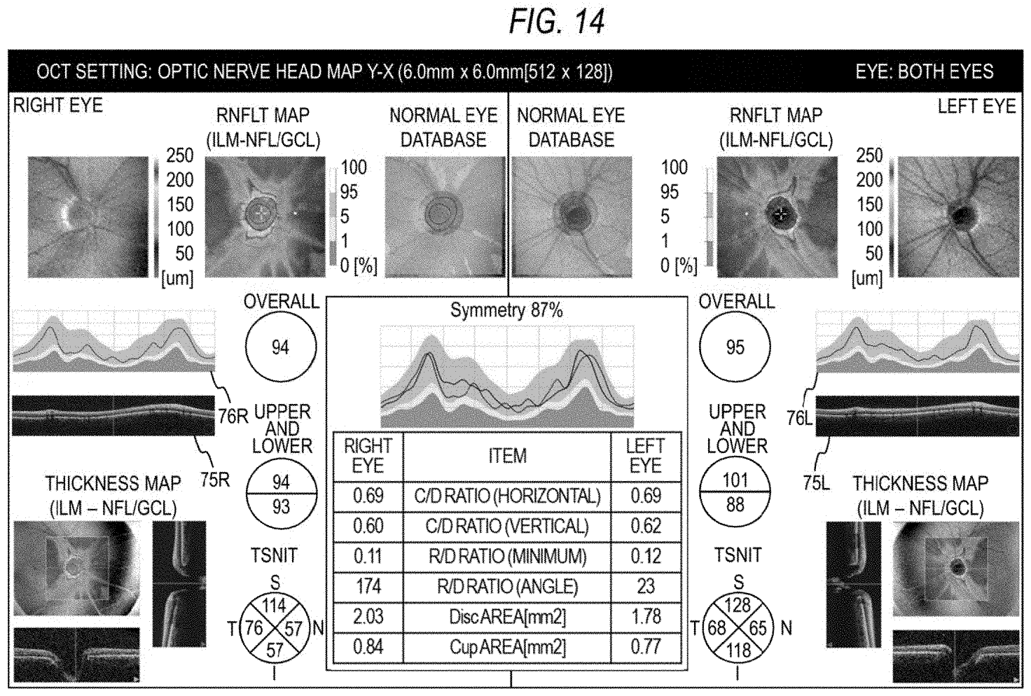

The CPU 3 displays the layer thickness graph generated in S23 on the display device 8 in a state of comparison with the data of the normal eye (S24). FIG. 14 shows an example of a display method for two-dimensional tomographic images 75R and 75L and layer thickness graphs 76R and 76L. In the example shown in FIG. 14, the two-dimensional tomographic images 75R and 75L extracted in S22 are respectively displayed for the right eye and the left eye of the subject. The layer thickness graphs 76R and 76L generated in S23 are displayed to be arranged with the corresponding two-dimensional tomographic images 75R and 75L. In the layer thickness graphs 76R and 76L, a range of data for normal eyes is displayed together with a graph representing a thickness of a specific layer analyzed on the basis of the two-dimensional tomographic images 75R and 75L. Therefore, the user can appropriately ascertain a state of the subject eye.

(Site Detection Processing)

The site detection processing performed by the fundus image processing apparatus 1 will be described with reference to FIGS. 15A to 17. The site detection processing is performed by the CPU 3 according to the fundus image processing program stored in the storage device 4. In the site detection processing, the second site different from the first site is identified based on the degree of deviation of a probability distribution in a case where the mathematical model identifies the first site. As described above, in the present embodiment, the site identification processing is performed in a case where an approximate position of the optic nerve head is automatically detected as the second site.

In general, a plurality of layers and boundaries are normally present around the optic nerve head, but specific layers and boundaries are missing at the position of the optic nerve head. Specifically, at a position where the optic nerve head is present, the NFL is present, and layers and boundaries at positions deeper than the NFL are missing. Based on the above findings, in the site detection processing of the present embodiment, the optic nerve head is identified based on the degree of deviation of a probability distribution in a case where the mathematical model identifies a specific layer/boundary (layer/boundary at a position deeper than the NFL).

As shown in FIG. 15A, the CPU 3 acquires a fundus image of the subject eye for detection of the second site (the optic nerve head, in the present embodiment) (S31). In the present embodiment, the three-dimensional tomographic image 43 (refer to FIG. 5) of the fundus of the subject eye is acquired as a fundus image, and a second site is detected based on the three-dimensional tomographic image 43. Therefore, the second site is detected based on more data than in a case where the second site is detected from the two-dimensional fundus image. In a case where the three-dimensional tomographic image 43 has already been acquired in S1 in FIG. 8A, the processing in S31 may be omitted.

Next, the CPU 3 acquires a two-dimensional front image in a case where the fundus of which the three-dimensional tomographic image 43 acquired in S31 (or S1) is captured is viewed from the front (that is, the direction along the OCT measurement light) (S32). As an example, in S32 of the present embodiment, the Enface image 45 (refer to FIG. 5) generated based on the data of the three-dimensional tomographic image 43 acquired in S31 is acquired, as a two-dimensional front image. However, the two-dimensional front image may be an image (for example, a two-dimensional front image captured by the front observation optical system 23) captured on the basis of a principle different from the principle of capturing the three-dimensional tomographic image 43.

The CPU 3 acquires an auxiliary identification result of the second site (the optic nerve head in the present embodiment), based on the two-dimensional front image acquired in S32 (S33). A method of auxiliary identification of the second site for the two-dimensional front image may be selected as appropriate. In the present embodiment, the CPU 3 identifies the optic nerve head by performing known image processing on the two-dimensional front image.

The CPU 3 extracts a part in which the second site (the optic nerve head in the present embodiment) is included with a high probability, from the entire three-dimensional tomographic image 43 acquired in S31 (or S1), based on the auxiliary identification result acquired in S33 (S34). As a result, an amount of subsequent processing is reduced, and thus the second site is detected more appropriately.

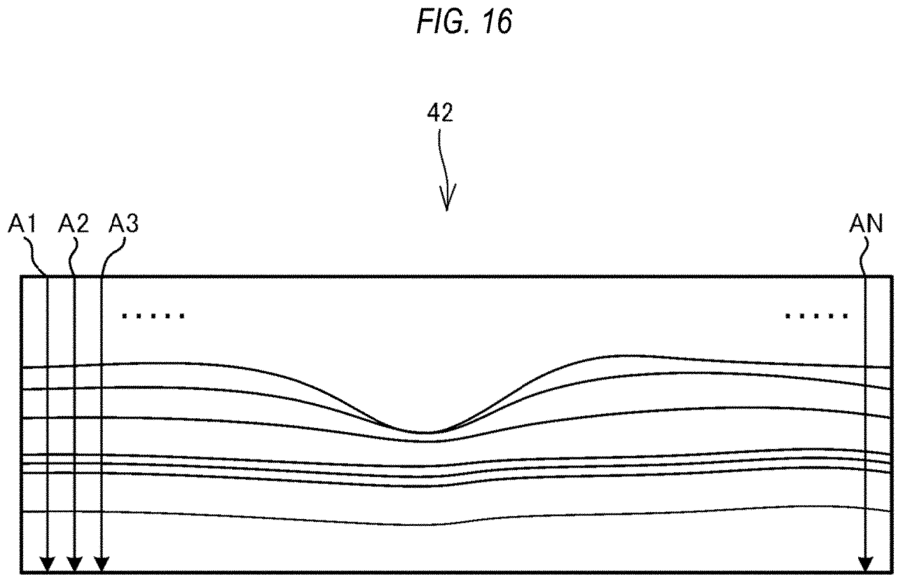

The CPU 3 extracts a T-th two-dimensional tomographic image (where an initial value of T is “1”), from among the plurality of two-dimensional tomographic images configuring the three-dimensional tomographic image extracted in S34 (S36). FIG. 16 shows an example of the extracted two-dimensional tomographic image 42. The two-dimensional tomographic image 42 shows a plurality of layers/boundaries in the fundus of the subject eye. A plurality of one-dimensional regions A1 to AN are set in the two-dimensional tomographic image 42. In the present embodiment, the one-dimensional regions A1 to AN set in the two-dimensional tomographic image 42 extend along an axis intersecting a specific layer/boundary. Specifically, the one-dimensional regions A1 to AN of the present embodiment correspond to a plurality (N) of respective A-scan regions configuring the two-dimensional tomographic image 42 captured by the OCT apparatus 10.

By inputting the T-th two-dimensional tomographic image into the mathematical model, the CPU 3 acquires a probability distribution of coordinates at which an M-th (where an initial value of M is “I”) layer/boundary is present in each of the plurality of one-dimensional regions A1 to AN, as a probability distribution for identifying the first site (specific layer/boundary) (S37). The CPU 3 acquires the degree of deviation of a probability distribution related to the M-th layer/boundary (S38). The degree of deviation is a difference in the probability distribution acquired in S37 with respect to the probability distribution in a case where the first site is accurately identified. In a one-dimensional region where the first site is present, the degree of deviation tends to be small. On the other hand, in a one-dimensional region where the first site is not present, the degree of deviation tends to be large. This tendency is likely to appear regardless of the presence or absence of an cyc disease or the like.

In the present embodiment, the entropy of the probability distribution P is calculated as the degree of deviation. The entropy is given by the following (Equation 1). The entropy H(P) takes a value of 0≤H(P)≤log (number of events), and becomes a smaller value as the probability distribution P is biased. That is, the smaller the entropy H(P), the higher the identification accuracy of the first site tends to be. The entropy of the probability distribution in a case where the first site is accurately identified is 0.

H(P)=−Σp log(p) (Equation 1)

Next, the CPU 3 determines whether or not the degree of deviation of all layers/boundaries to be identified in the T-th two-dimensional tomographic image has been acquired (S40). In a case where the degree of deviation of some layers/boundaries is not acquired (S40: NO), “1” is added to the order M of layers/boundaries (S41), the processing returns to S37, and the degree of deviation of the next layer/boundary is acquired (S37, S38). In a case where the degree of deviation of all layers/boundaries has been acquired (S40: YES), the CPU 3 stores the degree of deviation of the T-th two-dimensional tomographic image in the storage device 4 (S42).

Next, the CPU 3 determines whether or not the degree of deviation of all the two-dimensional tomographic images configuring the three-dimensional tomographic image has been acquired (S44). In a case where the degree of deviation of some two-dimensional tomographic images is not acquired yet (S44: NO), “1” is added to the order T of the two-dimensional tomographic images (S45), the processing returns to S36, and the degree of deviation of the next two-dimensional tomographic image is acquired (S36 to S42).

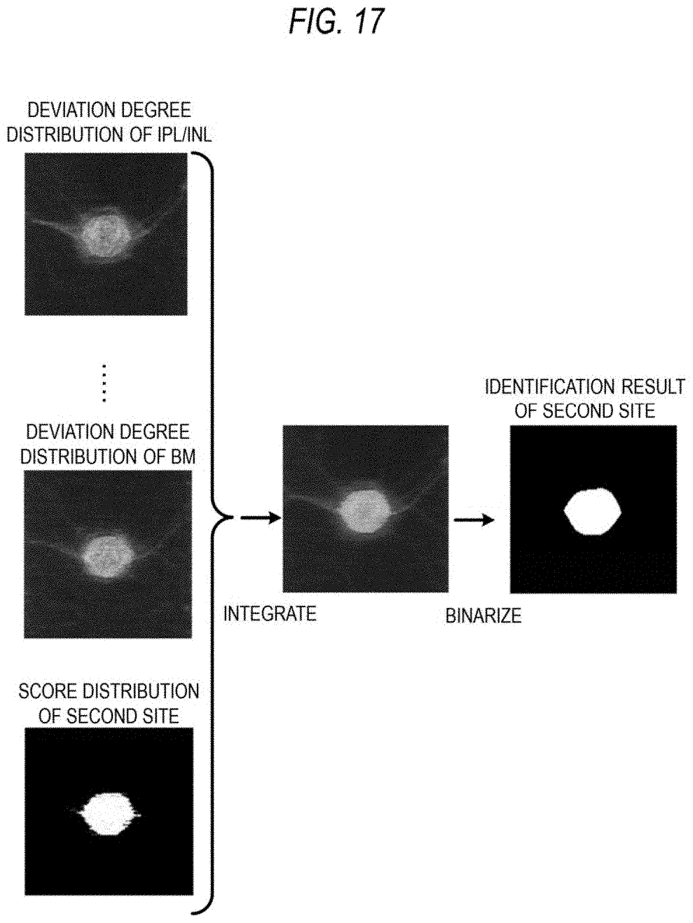

In a case where the degree of deviation of all the two-dimensional tomographic images has been acquired (S44: YES), the CPU 3 acquires a two-dimensional distribution of a magnitude of the degree of deviation (hereinafter, simply referred to as a “deviation degree distribution”) in a case where the fundus is viewed from the front (S47). In the present embodiment, as shown in FIG. 17, the CPU 3 acquires a deviation degree distribution of a specific layer/boundary among a plurality of layers/boundaries in the fundus. Specifically, at the position where the optic nerve head is present, the NFL is present, and layers and boundaries at positions deeper than the NFL are missing. Therefore, at the position where the optic nerve head is present, the degree of deviation related to identification of layers and boundaries at positions deeper than the NFL is higher than that at the position where the optic nerve head is not present. Therefore, in S47 of the present embodiment, in order to identify the optic nerve head with high accuracy, deviation degree distributions of layers/boundaries (specifically, a plurality of layers/boundaries including IPL/INL and the BM) at positions deeper than the NFL are acquired. In the deviation degree distribution shown in FIG. 17, a site having a high degree of deviation is represented in a bright color.

The CPU 3 acquires a distribution of scores indicating a probability that each site (each A-scan image) is the second site (hereinafter, referred to as a “score distribution of the second site”) (S48). As described above, the score distribution of the second site is output from the mathematical model together with the identification result of the first site.

Next, the CPU 3 generates an identification result of the second site based on the degree of deviation in a case where the mathematical model identifies the first site (S49). In the present embodiment, as shown in FIG. 17, the CPU 3 integrates (adds) the deviation degree distribution of the layer/boundary at a position deeper than the NFL and the score distribution of the second site. The CPU 3 generates the identification result of the second site by performing a binarization processing on the integrated distribution. In a case of integrating the deviation degree distribution and the score distribution, any weighting may be performed.

### Modification Examples

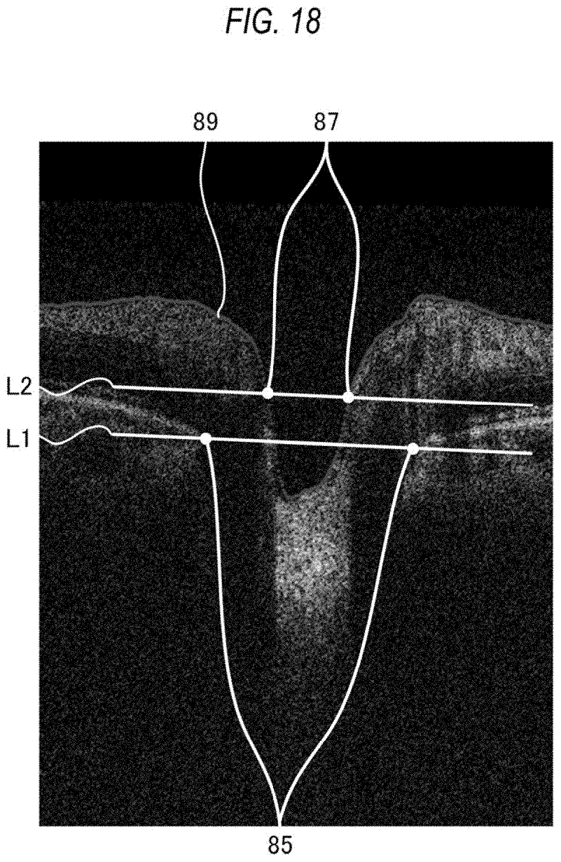

The techniques disclosed in the above embodiment are merely examples. Therefore, the techniques exemplified in the above embodiment may be changed. For example, the CPU 3 may detect a structure other than the optic nerve head in the fundus, based on a detection result of the end of the optic nerve head detected through the fundus image processing (refer to FIGS. 8A and 8B). In the example shown in FIG. 18, the CPU 3 detects a position of an optic disk recess (Cup) 87 based on the position of the BMO 85 detected through the fundus image processing. Specifically, the CPU 3 sets a straight line L2 that is parallel to a reference straight line L1 that passes through the pair of detected BMOs 85 and is separated from the reference straight line L1 toward the surface side of the retina by a predetermined distance. The CPU 3 detects a position where the set straight line L2 and an internal limiting membrane (ILM) 89 in the fundus image intersect, as a position of the Cup 87. The CPU 3 detects the shortest distance between the position of the BMO 85 detected through the fundus image processing and the ILM 89 in the fundus image, as the minimum thickness (minimum rim width) of the nerve fiber layer. According to the fundus image processing, a position of the end of the optic nerve head is detected with high accuracy. Therefore, a structure other than the optic nerve head is detected based on the detected position of the end of the optic nerve head, and thus the structure other than the optic nerve head is also detected with high accuracy.

In S3 of the fundus image processing (refer to FIG. 8A) of the above embodiment, the site identification processing shown in FIGS. 15A and 15B is used for automatically detecting the optic nerve head. However, the processing in S3 in FIG. 8A may also be changed. For example, the CPU 3 may automatically detect a position of the optic nerve head, based on a two-dimensional front image (that is, a two-dimensional image in a case of being viewed from the direction along the optical axis of the OCT measurement light) of the fundus of the subject eye that is an examination target. In this case, the CPU 3 may detect a position of the optic nerve head by performing known image processing on the two-dimensional front image. The CPU 3 may detect a position of the optic nerve head by inputting the two-dimensional front image into a mathematical model that detects and outputs the position of the optic nerve head. As the two-dimensional front image, various images such as the above-described Enface image 45, fundus camera image, or SLO image may be used.

In S10 in FIG. 8A, a specific method of setting the radial pattern 60 centered on the reference position RP may also be changed as appropriate. For example, the CPU 3 may acquire information regarding a position of a fundus blood vessel in the measurement region 40 of which a three-dimensional tomographic image is captured. The CPU 3 may adjust at least any one of the angle of the overall radial pattern 60, an angle of at least any one of the lines 61 included in the radial pattern 60, a length of the line 61, the number of lines 61, and the like, to reduce an amount of overlap between the lines 61 of the radial pattern 60 and the fundus blood vessels as much as possible. In this case, it is appropriately suppressed that the detection accuracy of the end of the optic nerve head deteriorates due to the presence of the fundus blood vessel. The CPU 3 may adjust at least any one of the angle of the overall radial pattern 60, an angle of at least any one of the lines 61 included in the radial pattern 60, a length of the line 61, the number of lines 61, and the like according to an instruction input from a user that has checked the fundus image. In this case, the detection accuracy of the end of the optic nerve head is further improved.

In the fundus image processing of the above embodiment (refer to FIGS. 8A and 8B), as the reference position RP becomes closer to a center position of the actual optic nerve head, a position of the end of the optic nerve head in each of the plurality of two-dimensional tomographic images 64 extracted according to the radial pattern 60 becomes more approximate, and thus the detection accuracy of the annular end of the optic nerve head becomes higher. The reference position RP set in S6 and S7 may be far from an actual center position of the optic nerve head. Therefore, in a case where the CPU 3 has performed the detection processing of the end of the optic nerve head shown in S3 to S14 only once, the CPU 3 may reset the reference position RP at the center position specified in S14 after performing the processing in S14, and perform the processing in S10 to S14 again. The center position of the optic nerve head specified in S14 tends to be more accurate than the center position detected through in the processing in S3 or the like. Therefore, the end of the optic nerve head is detected again with the center position of the optic nerve head specified in S14 as the reference position RP, and thus the detection accuracy is further improved. The number of times the processing in S10 to S14 are repeatedly performed may be set as appropriate. For example, the CPU 3 may perform the processing in and after S21 in a case where a center position of the optic nerve head specified a plurality of times in S14 converges within a certain range.

In the above embodiment, the site identification processing shown in FIGS. 15A and 15B is performed as a part of the fundus image processing shown in FIGS. 8A and 8B. However, the site identification processing shown in FIGS. 15A and 15B may be performed independently. In this case, it is also possible to detect a site other than the optic nerve head in the fundus image through the site identification processing. In general, a plurality of layers and boundaries are normally present around the fovea, but specific layers and boundaries are missing at the position of the fovea. Specifically, at the position where the fovea is present, the RPE, Bruch's membrane, and the like are present, and layers and boundaries nearer to the surface side of the retina than the RPE are missing. On the basis of the above findings, the fundus image processing apparatus 1 may identify the fovea (second site) based on the degree of deviation of a probability distribution in a case where the mathematical model identifies a layer/boundary (first site) nearer to the surface side of the retina than the RPE. In this case, in S37 to S47 in FIGS. 15A and 15B, a deviation degree distribution of at least any one of the layers/boundaries nearer to the surface side than the RPE is acquired, as the degree of deviation related to analysis of the first site. In S49, the fovea is identified as the second site. As a result, the fovea is identified with high accuracy.

At the position where the fundus blood vessel (second site) is present, the measurement light is blocked by the fundus blood vessel, and thus an imaging state of a layer/boundary (first site) at a position deeper than the fundus blood vessel tends to deteriorate. Therefore, at the position where the fundus blood vessel is present, the degree of deviation related to identification of the layer/boundary at the position deeper than the fundus blood vessel is larger than that at a position where the fundus blood vessel is not present. On the basis of the above findings, in S47, a deviation degree distribution of at least any one of layers/boundaries at positions deeper than the fundus blood vessel may be acquired, as the degree of deviation related to analysis of the first site. In S49, a site having the degree of deviation more than a threshold value may be identified as a site (second site) of the fundus blood vessel.

For example, only some of the plurality of techniques exemplified in the above embodiment may be performed. For example, in S33 and S34 (refer to FIG. 15A) of the above embodiment, the auxiliary identification result of the second site performed based on the two-dimensional front image is used. However, the second site may be identified without using the auxiliary identification result. In S48 and S49 (refer to FIG. 158) of the above embodiment, the score distribution of the second site is used. However, the second site may be identified without using the score distribution of the second site.

The processing of acquiring a three-dimensional tomographic image in S1 in FIG. 8A is an example of “image acquisition processing”. The processing of setting a reference position in S6 and S7 in FIG. 8A is an example of “reference position setting processing”. The processing of setting a radial pattern in S10 in FIG. 8A is an example of “radial pattern setting processing”. The processing of extracting a two-dimensional tomographic image in S11 in FIG. 8A is an example of “image extraction processing”. The processing of detecting a position of the end of the optic nerve head in S12 in FIG. 8A and S13 in FIG. 8B is an example of “optic nerve head end detection processing”. The processing of performing image alignment in S2 in FIG. 8A is an example of “alignment processing”. The processing of automatically detecting a position of the optic nerve head in S3 in FIG. 8A is an example of “optic nerve head position detection processing”. The processing of specifying a center position of the optic nerve head in S14 in FIG. 8B is an example of “optic nerve head center specifying processing”. The processing of extracting a two-dimensional tomographic image in S22 in FIG. 8B is an example of “annular shape extraction processing”. The processing of outputting information regarding a two-dimensional tomographic image in S24 in FIG. 8B is an example of “output processing”.

The processing of acquiring a fundus image in S31 in FIG. 15A is an example of “image acquisition processing”. The processing of acquiring the degree of deviation in S37 to S47 in FIGS. 15A and 15B is an example of “deviation degree acquisition processing”. The processing of identifying a second site in S49 in FIG. 15B is an example of “site identification processing”. The processing of acquiring a two-dimensional front image in S32 in FIG. 15A is an example of “front image acquisition processing”. The processing of acquiring an auxiliary identification result in S33 in FIG. 15A is an example of “auxiliary identification result acquisition processing”.

What is claimed is:

1. A fundus image processing apparatus that processes a tomographic image of a fundus of a subject eye captured by an OCT apparatus, the fundus image processing apparatus comprising:
a controller configured to perform:
image acquisition processing of acquiring a three-dimensional tomographic image of the fundus of the subject eye, the three-dimensional tomographic image being captured by irradiating a two-dimensional measurement region extending in a direction intersecting an optical axis of OCT measurement light with the OCT measurement light;
reference position setting processing of setting a reference position in a region of an optic nerve head in the two-dimensional measurement region in which the three-dimensional tomographic image was captured;
radial pattern setting processing of setting a radial pattern with respect to the two-dimensional measurement region, the radial pattern being a line pattern extending radially around the reference position;
image extraction processing of extracting a two-dimensional tomographic image in each of a plurality of lines of the radial pattern set in the redial pattern setting processing, from the three-dimensional tomographic image; and
optic nerve head end detection processing of detecting a position of an end of the optic nerve head captured in the three-dimensional tomographic image, based on a plurality of the two-dimensional tomographic images extracted in the image extraction processing.

2. The fundus image processing apparatus according to claim 1,
wherein the controller is configured to further perform:
alignment processing of performing image alignment, in a direction along the optical axis of the OCT measurement light, of the three-dimensional tomographic image or the two-dimensional tomographic image extracted in the image extraction processing, and
in the optic nerve head end detection processing, the controller is configured to detect the position of the end of the optic nerve head, based on the two-dimensional tomographic image for which the image alignment was performed.

3. The fundus image processing apparatus according to claim 1,
wherein the controller is configured to further perform:
optic nerve head position detection processing of automatically detecting a position of the optic nerve head in the two-dimensional measurement region, based on an image of the fundus, and
in the reference position setting processing, the controller is configured to set the reference position at the position of the optic nerve head automatically detected in the optic nerve head position detection processing.

4. The fundus image processing apparatus according to claim 1,
wherein, in the optic nerve head end detection processing, the controller is configured to input the plurality of two-dimensional tomographic images extracted in the image extraction processing into a mathematical model, and acquire the position of the end of the optic nerve head captured in each of the plurality of two-dimensional tomographic images to detect the position of the end of the optic nerve head, the mathematical model being trained with using a machine learning algorithm and outputting a detection result of an end of an optic nerve head captured in an input two-dimensional tomographic image.

5. The fundus image processing apparatus according to claim 1,
wherein, in the optic nerve head end detection processing, the controller is configured to smooth detection results of a plurality of positions of the end of the optic nerve head detected based on the plurality of two-dimensional tomographic images, to detect a position of an annular end of the optic nerve head.

6. The fundus image processing apparatus according to claim 1,
wherein the controller is configured to further perform:
optic nerve head center specifying processing of specifying a center position of the optic nerve head, based on the position of the end of the optic nerve head detected in the optic nerve head end detection processing.

7. The fundus image processing apparatus according to claim 6,
wherein the controller is configured to set the center position of the optic nerve head specified in the optic nerve head center specifying processing as the reference position in the reference position setting processing, and again perform the reference position setting processing, the radial pattern setting processing, the image extraction processing, and the optic nerve head end detection processing.

8. The fundus image processing apparatus according to claim 6,
wherein the controller is configured to further perform:
annular shape extraction processing of extracting a two-dimensional tomographic image in an annular line pattern centered on the center position of the optic nerve head specified in the optic nerve head center specifying processing, from the three-dimensional tomographic image; and
output processing of outputting information regarding the two-dimensional tomographic image extracted in the annular shape extraction processing.

9. A non-transitory computer-readable storage medium storing a fundus image processing program executed by a fundus image processing apparatus that processes a tomographic image of a fundus of a subject eye captured by an OCT apparatus, the fundus image processing program being executed by a controller of the fundus image processing apparatus to cause the fundus image processing apparatus to perform:
image acquisition processing of acquiring a three-dimensional tomographic image of the fundus of the subject eye, the three-dimensional tomographic image captured by irradiating a two-dimensional measurement region extending in a direction intersecting an optical axis of OCT measurement light with the OCT measurement light;
reference position setting processing of setting a reference position in a region of an optic nerve head in the two-dimensional measurement region in which the three-dimensional tomographic image was captured;
radial pattern setting processing of setting a radial pattern with respect to the two-dimensional measurement region, the radial pattern being a line pattern extending radially around the reference position;
image extraction processing of extracting a two-dimensional tomographic image in each of a plurality of lines of the radial pattern set in the radial pattern setting processing, from the three-dimensional tomographic image; and
optic nerve head end detection processing of detecting a position of an end of the optic nerve head captured in the three-dimensional tomographic image, based on a plurality of the two-dimensional tomographic images extracted in the image extraction processing.
